(19) United States
(12) Patent Application Publication
(10) Pub. No.: US 2016/0120695 A1
(43) Pub. Date: May 5, 2016

(54) USE OF PEDF IN AN ENCAPSULATED CELL-BASED DELIVERY SYSTEM

(71) Applicant: Neurotech USA, Inc., Cumberland, RI (US)

(72) Inventors: Weng Tao, Lincoln, RI (US); Konrad Kauper, Sutton, MA (US); Paul Stabila, Coventry, RI (US); Vincent Ling, Walpole, MA (US)

(21) Appl. No.: 14/979,159

(22) Filed: Dec. 22, 2015

Related U.S. Application Data
(63) Continuation of application No. 12/899,026, filed on Oct. 6, 2010.
(60) Provisional application No. 61/249,787, filed on Oct. 8, 2009.

(51) Int. Cl. A61F 9/00

(52) U.S. Cl. CPC A61F 9/0017; A61F 2220/0008; A61F 2250/0068; A61F 2240/001

(57) ABSTRACT

The invention relates to a device for delivery of pigment epithelium derived factor (PEDF) to the eye utilizing encapsulated PEDF-secreting cells and related methods for the treatment and prevention of ophthalmic diseases and disorders.

### RELATED APPLICATIONS

This application is a continuation of U.S. application Ser. No. 12/899,026, filed on Oct. 6, 2010, which in turn claims priority to U.S. Provisional Application No. 61/249,787, filed Oct. 8, 2009, the contents of each of which are herein incorporated by reference in its entirety.

### FIELD OF THE INVENTION

The present invention relates to the use of pigment epithelium derived factor (PEDF), and biologically active variants thereof, in a delivery system utilizing encapsulated cells engineered to secrete PEDF, and related methods for the treatment of ophthalmic diseases and disorders using encapsulated PEDF-secreting cells.

### INCORPORATION BY REFERENCE OF SEQUENCE LISTING

The contents of the text file named “NETE-056_C01US 322144-2879_ST25.txt”, which was created on Dec. 22, 2015 and is 13 KB in size, are hereby incorporated by reference in their entireties.

### BACKGROUND OF THE INVENTION

Pigment epithelium derived factor (PEDF) was first identified in the conditioned medium of cultured fetal human retinal pigment epithelial cells as a 50-kDa protein having neurotrophic activity (Tombran-Tink et al., Invest. Ophthalmol. Vis. Sci., (1989) 30:1700-1707; Tombran-Tink et al., Exp. Eye Res., (1991) 53:411-414). PEDF induces extensive neuronal differentiation in retinoblastoma cells (Chader, Cell Different., (1987) 20:209-216). It is also a potent inhibitor of angiogenesis and neovascularization (Dawson et al, Science (1999), 285(5425):245-8, Maik-Rachline et al., Blood (2005) 105:670-678; U.S. Pat. No. 7,105,496) and has been reported to be an inhibitor of VEGF-induced vascular permeability (see PCT International Application Publication No. WO2005041887). PEDF is also useful for treatment of various retinal degenerative diseases. (See Tombran-Tink, Frontiers in Bioscience 10:2131-2149 (2005)).

PEDF has extensive sequence homology with the serpin gene family, many members of which are serine protease inhibitors. However PEDF has no serine protease activity. Thus, PEDF is a non-inhibitory serpin having both neuroprotective and anti-angiogenic actions. (See Tombran-Tink, Frontiers in Bioscience 10:2131-2149 (2005)). The anti-angiogenic activity of PEDF makes it a promising candidate for therapy of a number of diseases and disorders characterized by aberrant neovascularization. For example, PEDF demonstrated inhibition of neovascularization (up to 85%) in three murine disease models, the laser-induced choroidal neovascularization model, the VEGF transgenic model, and the retinopathy of prematurity model (see discussion in Rasmussen et al., Hum Gene Ther. (2001) 12:2029-2032). In addition, PEDF has shown efficacy in a Phase I clinical trial in humans for the treatment of age-related macular degeneration (Campochiaro et al., Hum Gene Ther. (2006) 17:167-176).

Successful treatment of ophthalmic diseases and disorders depends upon the ability to deliver the desired therapeutic agent(s) to the eye, or to a particular region of the eye, in an amount sufficient to produce the desired biological activity. Protein or peptide-based therapeutics in particular have proven difficult to administer to the eye. Oral administration is typically not effective to provide the desired dosage to the eye. Topical administration of liquids, gels, or ointments tends to be ineffective for protein or peptide-based therapeutics which are not easily formulated for topical delivery and which may be unable to cross the cornea. In addition, topical formulations tend to be ineffective for delivery to the sclera, vitreous, or posterior segment of the eye. Direct intraocular injection, for example, into the vitreous, has resulted in undesirable side effects such as an increased incidence of retinal macrophages and cataracts (see La Vail et al., Proc. Natl. Acad. Sci. U.S.A. 1992, 89:11249).

Another option for ophthalmic delivery of therapeutic agents is the use of an intraocular insert. See e.g., U.S. Pat. Nos. 3,828,777; 4,343,787; 4,730,013; 4,164,559; 5,395,618; 5,466,233; and Anand, R. et al., Arch. Ophthalmol. 1993 111:223. However, release of proteins from such devices (or other erodible or nonerodible polymers) can be sustained for only short periods of time due to protein instability, making them unsuitable for delivery of most, if not all, protein molecules.

The implantation of encapsulated cells engineered to produce the therapeutic agent is an attractive alternative for the delivery of such agents to eye, especially those whose efficacy depends on their reaching regions of the eye not easily accessible by topical administration.

The delivery of desired growth factors using encapsulated cells has shown efficacy in pre-clinical and clinical studies. For example, ciliary neurotrophic factor (CNTF) was delivered continuously with therapeutic efficacy in a rodent model (Emerich et al., J. Neurosci. (1996) 16:5168-5181) and the safety of chronic CNTF delivery into the human central nervous system (CNS) with polymer-encapsulated cells has been demonstrated (Aebischer et al., Hum. Gene Ther. (1996) 7:851-860; Aebischer et al., Nature Med. (1996) 2:696-699). In addition CNTF has been successfully delivered to the human eye using encapsulated cells (Sieving et al., Proc Natl Acad Sci (USA) (2006) 103(10):3896-901).

### SUMMARY OF THE INVENTION

The present invention relates to the use of pigment epithelium derived factor (PEDF), and biologically active variants thereof, in a delivery system utilizing encapsulated cells engineered to secrete PEDF, and methods of use for the treatment of ophthalmic diseases and disorders.

The invention provides implantable cell culture devices containing a core that contains one or more ARPE-19 cells that are genetically engineered to secrete PEDF and a semipermeable membrane surrounding the core, wherein the membrane permits the diffusion of PEDF therethrough. Those skilled in the art will recognize that the cells may secrete a PEDF variant, for example a PEDF variant having the amino acid sequence of SEQ ID NO:1 or a biologically active fragment of PEDF.

The devices of the invention may also contain a matrix (e.g., a hydrogel matrix or extracellular matrix disposed within the semipermeable membrane. In some embodiments, the hydrogel is alginate cross-linked with a multivalent ion. In other embodiments, the matrix is a plurality of monofilaments, wherein said monofilaments are twisted into a yarn or woven into a mesh or twisted into a yarn that is in non-woven strands, wherein the cells or tissue are distributed thereon. For example, the filamentous cell-supporting matrix comprises a biocompatible material selected from acrylic, polyester, polyethylene, polypropylene polyacetonitrile, polyethylene terephthalate, nylon, polyamides, polyurethanes, polybutester, silk, cotton, chitin, carbon, and/or biocompatible metals.

The devices of the invention may also contain a tether anchor. For example, the tether anchor may contain an anchor loop that is adapted for anchoring the device to an ocular structure.

Moreover, those skilled in the art will recognize that the devices of the invention are suitable for implantation into the eye. For example, the devices can be implanted, inserted, or used in the vitreous, the aqueous humor, the Subtenon's space, the periocular space, the posterior chamber, or the anterior chamber of the eye.

In various embodiments, the jacket of the devices of the invention is a permselective, immunoisolatory membrane. By way of non-limiting example, the jacket can be an ultrafiltration membrane or a microfiltration membrane. In addition, the jacket can be formed of a non-porous membrane material such as a hydrogel or a polyurethane. Suitable materials for the semipermeable membrane include, but are not limited to polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), and/or derivatives, copolymers and mixtures thereof. In some embodiments, the semipermeable membrane has a molecular weight cutoff of from 1 to 1500 kilodaltons.

Those skilled in the art will recognize that the devices of the invention may be configured as a hollow fiber or a flat sheet. For example, the device may be a hollow fiber (e.g., a poly sulfone hollow fiber) having an outer diameter between 200 and 350 μm and a length of between 0.4 mm and 6 mm.

In some embodiments, at least one additional biologically active molecule (i.e., from a cellular or a non-cellular source) is delivered from the devices described herein. For example, the at least one additional biologically active molecule is produced by one or more genetically engineered ARPE-19 cell in the core.

The devices of the invention may have a core volume of between 1 and 3 Alternatively, micronized devices according to the invention may have a core volume of between 0.05 and 0.1 μl. By way of non-limiting example, the capsule may contain from about 10⁴ to 10⁷ cells.

Also provided are methods for treating an ophthalmic diseases or disorders characterized by retinal degeneration, neovascularization fluid accumulation in the eye, or any combination thereof, in a subject in need of such treatment (e.g., a human), by implanting any of the implantable cell culture devices of the invention into the eye (e.g., intraocularly or periocularly) of the subject and allowing PEDF to diffuse from the device into the eye, thereby treating the disease or disorder. Likewise, any of the devices disclosed herein can be used in the treatment or management of such ophthalmic diseases or disorders. By way of non-limiting example, the ophthalmic disease or disorder is age-related macular degeneration, retinitis pigmentosa, diabetic macular edema, or diabetic retinopathy. For example, in such methods, between 0.1 pg and 1000 μg per eye per patient per day of PEDF diffuses into the eye.

The invention also provides methods for inhibiting neural or retinal degradation or degeneration in a host comprising implanting (e.g., intraocularly or periocularly) any of the cell culture devices of the invention into the eye of a host, wherein the device secretes a therapeutically effective amount of PEDF into the eye, thereby allowing PEDF to function as a neurotrophic or neuroprotective agent.

In other embodiments, the invention provides methods of delivering PEDF to a recipient host by implanting the implantable cell culture devices of the invention into a target region of the recipient host, wherein the encapsulated one or more ARPE-19 cells secrete PEDF at the target region. By way of non-limiting example, suitable target regions include, but are not limited to, the central nervous system, including the brain, ventricle, spinal cord, and the aqueous and vitreous humors of the eye. In such methods, between 0.1 pg and 1000 μg per patient per day of PEDF diffuses into the target region.

The invention also provides methods for inhibiting vasopermeability associated with angiogenesis, retinal disease, or a combination thereof in a host comprising implanting the cell culture device of the invention into the eye of a host, wherein the device secretes therapeutically effective amount of PEDF into the eye, thereby allowing PEDF to inhibit vasopermeability. (See Liu et al., Proc Natl Acad Sci (USA 101(17):6605-10 (2004) (incorporated herein by reference)).

The invention further provides methods for making the implantable cell culture devices of the invention by genetically engineering at least one ARPE-19 cell to secrete a PEDF polypeptide encoded by the nucleic acid sequence of SEQ ID NO:2 or SEQ ID NO:4, and encapsulating the genetically modified ARPE-19 cells within a semipermeable membrane, wherein said membrane allows the diffusion of PEDF therethrough. Alternatively, the implantable cell culture devices of the invention can be made by genetically engineering at least one ARPE-19 cell to secrete a PEDF polypeptide comprising the amino acid sequence of SEQ ID NO:1, and encapsulating the genetically modified ARPE-19 cells within a semipermeable membrane, wherein said membrane allows the diffusion of PEDF therethrough.

### BRIEF DESCRIPTION OF THE FIGURES

FIG. 1 shows the sequence of the pKan2 expression vector.

FIG. 2 is a Western blot of secreted recombinant human PEDF from stably transfected ARPE-19 cells. Conditioned media from stably transfected ARPE-19 expressing recombinant human PEDF was subjected to LDS-PAGE, transferred to PVDF membrane, which was then probed for PEDF. The primary antibody was mouse anti-human PEDF monoclonal (Millipore/Chemicon, Billerica, Mass.) diluted 1:500. The secondary antibody was donkey anti-mouse HRP-conjugated polyclonal antibody diluted 1:2000 (Jackson ImmunoResearch Laboratories, Westgrove, Pa.). Bands were visualized using TMB colorimetric substrate (KPL Inc., Gaitherburgh, Md.). Soluble PEDF migrated as a doublet of approximately 50 kD (arrow). Abbreviations: CM—conditioned media from PEDF stable cell line; rPEDF-recombinant human PEDF (BioProducts Maryland, Middletown, Md.); MW—Rainbow high molecular weight protein marker (GE Healthcare Life Sciences, Piscataway, N.J.).

FIG. 3 is a chart showing the change in Best Corrected Visual Acuity (BCVA) at baseline, 1 month, 3 months, 4 months, and 6 months post-implant. In this figure, T represents the NT-502 treated patients; C represents the control (Focal Laser); and Δ represents the change from baseline.

FIG. 4 is a graph showing the mean change in BCVA at baseline, 1 month, 3 months, 4 months, and 6 months for NT-502 treated patients and for laser treated patients.

FIG. 5 is a graph showing the change in BCVA for NT-502 and laser treated patients.

FIG. 6 shows Oscillatory Potentials (OP) results for 1 patient at baseline and at 6 months post-implant.

FIGS. 7A and 7B are a series of fundus photographs for two patients (Case 002 and Case 003) at baseline, month 1, month 3, and month 6. As shown in the baseline photo, both patients showed significant amounts of hard exudates in the eye. Following NT-502 treatment, over time, the hard exudates began to breakdown and were absorbed.

### DETAILED DESCRIPTION OF THE INVENTION

The present invention provides for the delivery of PEDF intraocularly (e.g., in the anterior chamber, posterior chamber, or vitreous of the eye) or periocularly (e.g., within or beneath Tenon's capsule), or both, utilizing encapsulated cells. The invention also provides methods for the treatment and prevention of ophthalmic diseases and disorders by delivering to a subject in need thereof an effective amount of PEDF utilizing encapsulated cells.

Cells that secrete PEDF can be encapsulated in a semipermeable membrane which allows for the diffusion of nutrients to the cells and also allows the secreted cellular products and waste materials to diffuse away from the cells. In some cases, the membrane may also serve to immunoisolate the cells by blocking the cellular and molecular effectors of immunological rejection. The use of immunoisolatory membranes allows for the implantation of allogeneic and xenogeneic cells into an individual without the use of immunosuppression. See e.g., U.S. Pat. No. 6,299,895. Encapsulated cells can be implanted directly into the region of the eye where the therapeutic agent is needed and provide continuous, long-term, low-level delivery of the desired therapeutic agent. This method also eliminates the risk of tumor formation from the implantation of naked cells or viruses engineered to produce the therapeutic agent, and decreases the risk of infection, since only a single penetration into the target site is required for continuous delivery.

A device containing encapsulated cells may also include a hydrogel matrix or other suitable three dimensional scaffold for enhancing cell viability and a tether which aids in retrieval of the device (see WO 92/19195). The cell-containing membrane may also have external supports for connecting a plurality of cell-containing tubular membranes (see WO 91/00119). The device may have a rigid or semi-rigid support structure (see WO 93/21902). The device may also take the form of a capsule comprising a semipermeable membrane (see U.S. Pat. No. 6,299,895).

The use of encapsulated cells provides numerous advantages over other delivery routes. For example, the therapeutic agent can be delivered directly to an intraocular or periocular region of the eye, reducing side effects from less targeted methods of delivery. In addition, relatively small doses (nanogram or low microgram quantities rather than milligrams) can be delivered compared with topical applications, also reducing the side effects associated with higher topical doses. A further advantage of encapsulated cells is that the cells continuously produce the therapeutic agent, avoiding the fluctuation in dose that characterizes delivery by injection. Finally, the use of encapsulated cells provides for a less invasive method of delivery than many prior art devices and surgical techniques, which result in a large number of retinal detachments.

The present invention provides an encapsulated cell delivery system comprising PEDF-secreting cells contained within a capsule. The encapsulated cells express a polynucleotide encoding PEDF and secrete PEDF into the extracellular environment in a therapeutically effective amount. Preferably, the amount is from about 1 ng to about 1000 ng (e.g., about 1, 2, 3, 4, 5, 6, 7, 8, 9, 10, 25, 50, 75, 100, 250, 500, 750, or 1000 ng) PEDF to the eye per capsule. The PEDF can be the full-length polypeptide of 418 amino acids or a biologically active fragment or variant thereof. Moreover, polynucleotides encoding PEDF can also be used.

Suitable cell types include any cell which produces PEDF in sufficient quantities to provide a therapeutically effective amount of PEDF to the eye. Preferably, the cells are ARPE-19 cells. However, those skilled in the art will recognize that any other suitable cell type can also be used in accordance with the methods and devices described herein.

In some embodiments, the capsule or device has a core containing the cells, either suspended in a liquid medium or immobilized within an immobilizing matrix or scaffold, and the capsule is enclosed by a semipermeable matrix or membrane “jacket” that does not contain cells. Preferably, the jacket is selectively permeable to control the diffusion of molecules into and out of the capsule based on molecular weight. The molecular weight cutoff of the jacket is chosen to allow easy diffusion of PEDF out of the capsule and into the surrounding tissue into which the capsule is implanted. The jacket also forms a barrier which prevents contact between the encapsulated cells and cells of the host immune system.

Ophthalmic diseases and disorders that can be treated or prevented using the encapsulated PEDF-secreting cells of the invention include those characterized by neovascularization and/or accumulation of fluid within the layers of the eye and within the vitreal cavity. Those skilled in the art will recognize that neovascularization requires angiogenesis. Thus, any diseases or disorders characterized by neovascularization can be treated with PEDF, which is an inhibitor of angiogenesis.

Moreover, vascular leakage can cause retinal detachment, degeneration of sensory cells of the eye, increased intraocular pressure, and inflammation, all of which adversely affect vision and the general health of the eye. A key factor in the regulation of vascular permeability is vascular endothelial growth factor (VEGF). As an inhibitor of VEGF-induced vascular permeability, PEDF is useful for the treatment of ophthalmic conditions characterized by the accumulation of fluid within the eye. Thus, the skilled artisan will recognize that certain ophthalmic diseases and disorders are characterized both by neovascularization and vascular leakage leading to fluid accumulation in the eye. Specific ophthalmic diseases and disorders that can be treated or prevented according to the methods of the invention include, but are not limited to, ocular tumors such as retinoblastoma, retinitis pigmentosa, diabetic retinopathies, proliferative retinopathies, retinopathy of prematurity, retinal vascular diseases, vascular anomalies, choroidal disorders, choroidal neovascularization, neovascular glaucoma, glaucoma, macular edema (e.g., diabetic macular edema), retinal edema (e.g., diabetic retinal edema), central serous chorioretinopathy, macular degeneration, and retinal detachment.

As used herein, the terms “individual” or “recipient” or “host” are used interchangeably to refer to a human or an animal subject.

A “biologically active molecule” (“BAM”) is a substance that is capable of exerting a biologically useful effect upon the body of an individual in whom a device of the present invention is implanted. For example, PEDF is an example of a suitable BAM.

The terms “capsule” and “device” and “vehicle” are used interchangeably herein to refer to the ECT devices of the invention.

Unless otherwise specified, the term “cells” means cells in any form, including, but not limited to, cells retained in tissue, cell clusters, and individually isolated cells.

As used herein a “biocompatible capsule” or “biocompatible device” or “biocompatible vehicle” means that the capsule or device or vehicle, upon implantation in an individual, does not elicit a detrimental host response sufficient to result in the rejection of the capsule or to render it inoperable, for example through degradation.

As used herein an “immunoisolatory capsule” or “immunoisolatory device” or “immunoisolatory vehicle” means that the capsule upon implantation into an individual, minimizes the deleterious effects of the host's immune system on the cells within its core.

As used herein “long-term, stable expression of a biologically active molecule” means the continued production of a biologically active molecule at a level sufficient to maintain its useful biological activity for periods greater than one month, preferably greater than three months and most preferably greater than six months. Implants of the devices and the contents thereof are able to retain functionality for greater than three months in vivo and in many cases for longer than a year.

The “semi-permeable” nature of the jacket membrane surrounding the core permits molecules produced by the cells (e.g., metabolites, nutrients and/or therapeutic substances) to diffuse from the device into the surrounding host eye tissue, but is sufficiently impermeable to protect the cells in the core from detrimental immunological attack by the host. In addition, those skilled in the art will recognize that the “semi-permeable” nature of the jacket is that the pore restriction prevents the escape of the encapsulated cells.

For immunoisolatory capsules, jacket nominal molecular weight cutoff (MWCO) values up to 1000 kD are contemplated. However, those skilled in the art will recognize that, in some cases, the MWCO may be greater than 1000 kD. In some embodiments, the MWCO is between 50-700 kD, e.g., between 70-300 kD. See, e.g., WO 92/19195.

The term “treatment” as used herein refers to a reduction, a partial improvement, amelioration, or a mitigation of at least one clinical symptom associated with the ophthalmic disease or disorder being treated. As used herein, the term “prevention” or “prophylaxis” refers to an inhibition or delay in the onset or progression of at least one clinical symptom associated with the ophthalmic disease or disorder to be prevented.

Moreover, the term “effective amount” as used herein refers to an amount that provides some improvement or benefit to the subject. In certain embodiments, an effective amount is an amount that provides some alleviation, mitigation, and/or decrease in at least one clinical symptom of the ophthalmic disease or disorder to be treated. In other embodiments, the effective amount is the amount that provides some inhibition or delay in the onset or progression of at least one clinical symptom associated with the ophthalmic disease or disorder to be prevented. The therapeutic effects need not be complete or curative, as long as some benefit is provided to the subject.

Likewise, as used herein, the term “subject” preferably refers to a human subject but may also refer to a non-human primate or other mammal preferably selected from among a mouse, a rat, a dog, a cat, a cow, a horse, or a pig.

Those skilled in the art will recognize that PEDF is a non-inhibitory serpin having both neuroprotective and anti-angiogenic actions. In particular, it is a potent and broadly acting neurotrophic factor that protects neurons from many CNS regions against a variety of neurodegenerative insults. Additionally, PEDF also functions as a natural inhibitor of angiogenesis. (See Tombran-Tink, Frontiers in Bioscience 10:2131-2149 (2005), herein incorporated by reference in its entirety).

The PEDF polypeptides for use in the present invention include the full-length polypeptide of 418 amino acids and biologically active fragments and variants thereof. Exemplary sequences of the full-length polypeptide include, without limitation, the sequence of GenBank Accession No. P36955 (Steele et al., Proc. Natl. Acad. Sci. U.S.A. (1993) 90:1526-1530) and other sequences known in the art (see e.g., U.S. Pat. No. 6,319,687 and PCT International Application Publication Nos. WO 95/33480 and WO 93/24529; see also WO 99/04806). In one embodiment, a biologically active fragment of PEDF is selected from a fragment consisting of amino acids 78-121, amino acids 44-77, amino acids 44-121, or amino acids 78-121 of the reference sequence (see PCT International Application Publication No. WO2005041887 and U.S. Patent Application Publication No. US20070087967). As used herein, the “reference sequence” refers to the sequence of GenBank Accession No. P36955.

A naturally occurring allelic variant of PEDF may also be used. Exemplary allelic variants of PEDF include, without limitation, a variant having a single amino acid substitution selected from the following: M72T and P132R (Koenekoop et al., Mol. Vis. (1999) 5:10; Gerhard et al., Genome Res. (2004) 14:2121-2127). Thus, in one embodiment, an allelic variant of PEDF has a substitution of the methionine at position 72 of the reference sequence with a threonine. In another embodiment, an allelic variant of PEDF has a substitution of the proline at position 132 of the reference sequence with an arginine.

Suitable PEDF polypeptides for use in the invention also include variant PEDF polypeptides having high sequence identity to the reference sequence which retain one or more biological activities selected from neurotrophic activity, neuroprotective activity, anti-angiogenic activity, anti-neovascularization activity, and anti-vasopermeability activity. For example, a suitable PEDF polypeptide retains one or more of the foregoing biological activities and has a sequence identity of at least 75%, at least 80%, at least 85%, at least 90%, or at least 95% compared to the reference sequence. Preferably, the variant PEDF polypeptide is at least 95%, at least 97%, at least 98%, or at least 99% identical to the reference sequence. Such variants may be formed by the insertion, deletion, or substitution of one or more amino acids in the reference sequence. Preferably, a substitution (other than a naturally occurring allelic variation) comprises a conservative substitution, meaning that a given amino acid is substituted with an amino acid having similar chemical properties. For example, positively-charged residues (H, K, and R) preferably are substituted with positively-charged residues; negatively-charged residues (D and E) preferably are substituted with negatively-charged residues; neutral polar residues (C, G, N, Q, S, T, and Y) preferably are substituted with neutral polar residues; and neutral non-polar residues (A, F, I, L, M, P, V, and W) preferably are substituted with neutral non-polar residues.

For example, the PEDF polypeptide for use in the invention comprises or consists of the amino acid sequence of SEQ ID NO:1.

M72T PEDF Variant
SEQ ID NO: 1
MQALVLLLCIGALLGHSSCQNPASPPEEGSPDPDSTGALVEEEDPFFKVP
VNKLAAAVSNFGYDLYRVRSSTSPTTNVLLSPLSVATALSALSLGAEQRT
ESIIHRALYYDLISSPDIHGTYKELLDTVTAPQKNLKSASRIVFEKKLRI
KSSFVAPLEKSYGTRPRVLTGNPRLDLQEINNWVQAQMKGKLARSTKEIP
DEISILLLGVAHFKGQWVTKFDSRKTSLEDFYLDEERTVRVPMMSDPKAV
LRYGLDSDLSCKIAQLPLTGSMSIIFFLPLKVTQNLTLIEESLTSEFIHD
IDRELKTVQAVLTVPKLKLSYEGEVTKSLQEMKLQSLFDSPDFSKITGKP
IKLTQVEHRAGFEWNEDGAGTTPSPGLQPAHLTFPLDYHLNQPFIFVLRD
TDTGALLFIGKILDPRGP.

The encapsulated cells of the invention express a polynucleotide encoding PEDF and secrete PEDF into the extracellular environment. PEDF can be the full-length polypeptide of 418 amino acids or a biologically active fragment or variant thereof as described above. The polynucleotide sequence encoding PEDF can be obtained from any source, e.g., isolated from nature, synthetically produced, or isolated from a genetically engineered organism. Preferably, the polynucleotide sequence encoding PEDF is one described in U.S. Pat. Nos. 5,840,686, 6,319,687, and 6,451,763; or in International Patent Applications WO 93/24529 and WO 95/33480. The skilled artisan will appreciate that, due to the degeneracy of the genetic code, more than one polynucleotide sequence can encode a given PEDF amino acid sequence.

For example, the PEDF polynucleotide comprises or consists of the cDNA sequence of SEQ ID NO:2.

M72T PEDF Variant cDNA
SEQ ID NO: 2
atgcaggccctggtgctactcctctgcattggagccctcctcgggcacagcagctgccagaaccctgccagccccccggaggagggctc
cccagaccccgacagcacaggggcgctggtggaggaggaggatcctttcttcaaagtccccgtgaacaagctggcagcggctgtctcca
acttcggctatgacctgtaccgggtgcgatccagcacgagccccacgaccaacgtgctcctgtctcctctcagtgtggccacggccctctcg
gccctctcgctgggagcggagcagcgaacagaatccatcattcaccgggctctctactatgacttgatcagcagcccagacatccatggta
cctataaggagctccttgacacggtcaccgccccccagaagaacctcaagagtgcctcccggatcgtctttgagaagaagctgcgcataaa
atccagctttgtggcacctctggaaaagtcatatgggaccaggcccagagtcctgacgggcaaccctcgcttggacctgcaagagatcaac
aactgggtgcaggcgcagatgaaagggaagctcgccaggtccacaaaggaaattcccgatgagatcagcattctccttctcggtgtggcg
cacttcaaggggcagtgggtaacaaagtttgactccagaaagacttccctcgaggatttctacttggatgaagagaggaccgtgagggtcc
ccatgatgtcggaccctaaggctgttttacgctatggcttggattcagatctcagctgcaagattgcccagctgcccttgaccggaagcatga
gtatcatcttcttcctgcccctgaaagtgacccagaatttgaccttgatagaggagagcctcacctccgagttcattcatgacatagaccgaga
actgaagaccgtgcaggcggtcctcactgtccccaagctgaagctgagttatgaaggcgaagtcaccaagtccctgcaggagatgaagct
gcaatccttgtttgattcaccagactttagcaagatcacaggcaaacccatcaagctgactcaggtggaacaccgggctggctttgagtgga
acgaggatggggegggaaccacccccagcccagggctgcagcctgcccacctcaccttcccgctggactatcaccttaaccagcctttca
tcttcgtactgagggacacagacacaggggcccttctcttcattggcaagattctggaccccaggggcccctaa

In another embodiment, the PEDF polynucleotide comprises or consists of the cDNA sequence of SEQ ID NO:4.

SEQ ID NO: 4
atgcaggccctggtgctactcctctgcattggagccctcctcgggcacagcagctgccagaaccctgccagccccccggaggagggctc
cccagaccccgacagcacaggggcgctggtggaggaggaggatcctttcttcaaagtecccgtgaacaagctggcagcggctgtctcca
acttcggctatgacctgtaccgggtgcgatccagcacgagccccacgaccaacgtgctcctgtctcctctcagtgtggccacggccctctcg
gccctctcgctgggagcggagcagcgaacagaatccatcattcaccgggctctctactatgacttgatcagcagcccagacatccatggta
cctataaggagctccttgacacggtcactgccccccagaagaacctcaagagtgcctcccggatcgtctttgagaagaagctgcgcataaa
atccagctttgtggcacctctggaaaagtcatatgggaccaggcccagagtcctgacgggcaaccctcgcttggacctgcaagagatcaac
aactgggtgcaggcgcagatgaaagggaagctcgccaggtccacaaaggaaattcccgatgagatcagcattctccttctcggtgtggcg
cacttcaaggggcagtgggtaacaaagtttgactccagaaagacttccctcgaggatttctacttggatgaagagaggaccgtgagggtcc
ccatgatgtcggaccctaaggctgttttacgctatggcttggattcagatctcagctgcaagattgcccagctgcccttgaccggaagcatga
gtatcatcttcttectgcccctgaaagtgacccagaatttgaccttgatagaggagagcctcacctccgagttcattcatgacatagaccgaga
actgaagaccgtgcaggcggtcctcactgtccccaagctgaagctgagttatgaaggcgaagtcaccaagtccctgcaggagatgaagct
gcaatccttgtttgattcaccagactttagcaagatcacaggcaaacccatcaagctgactcaggtggaacaccgggctggctttgagtgga
acgaggatggggcgggaaccacccccagcccagggctgcagcctgcccacctcaccttcccgctggactatcaccttaaccagcctttca
tcttcgtactgagggacacagacacaggggcccttctcttcattggcaagattctggaccccaggggcccctaa

Alternatively, the nucleic acid molecules may be the complement of such a nucleic acid molecule. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but preferably is double-stranded DNA.

An “isolated” nucleic acid molecule is one that is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A PEDF nucleic acid molecule (e.g., a nucleic acid molecule having the nucleotide sequence of SEQ ID NO: 2 or SEQ ID NO:4) encoding a polypeptide having the sequence of SEQ ID NO:1 or a complement thereof) can be isolated using standard molecular biology techniques and the sequence information provided herein. Using all or a portion of these nucleic acid sequences a hybridization probe, PEDF molecules can be isolated using standard hybridization and cloning techniques (e.g., as described in Sambrook et al., (eds.), Molecular Cloning: A Laboratory Manual 2nd Ed., Cold Spring Harbor Laboratory Press, Cold Spring Harbor, N. Y., 1989; and Ausubel, et al., (eds.), Current Protocols in Molecular Biology, John Wiley & Sons, New York, N.Y., 1993.)

Any PEDF nucleic acids can be amplified using cDNA, mRNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to PEDF nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment, an oligonucleotide comprising a nucleic acid molecule less than 100 nt in length would further comprise at least 6 contiguous nucleotides of SEQ ID NO: 2 or SEQ ID NO:4 or a complement thereof. Oligonucleotides may be chemically synthesized and may be used as probes.

In other embodiments, an isolated nucleic acid molecule comprises a nucleic acid molecule that is a complement of the PEDF nucleotide sequence. A nucleic acid molecule that is complementary to these nucleotide sequences is one that is sufficiently complementary to the nucleotide sequence that it can hydrogen bond with little or no mismatches, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, Van der Waals, hydrophobic interactions, etc. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Moreover, the nucleic acid molecule can comprise only a portion of the PEDF nucleic acid sequence, e.g., a fragment that can be used as a probe or primer or a fragment encoding a biologically active portion of PEDF. Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid. Derivatives or analogs include, but are not limited to, molecules comprising regions that are substantially homologous to the PEDF nucleic acids or proteins, in various embodiments, by at least about 30%, 50%, 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., Current Protocols in Molecular Biology, John Wiley & Sons, New York, N.Y., 1993, and below.

The invention further encompasses nucleic acid molecules that differ from the PEDF nucleotide sequence shown in SEQ ID NO:2 or SEQ ID NO:4 due to degeneracy of the genetic code and thus encode the same PEDF proteins as that encoded by the nucleotide sequence shown in SEQ ID NO: 2 or SEQ ID NO:4.

In another embodiment, an isolated PEDF nucleic acid molecule is at least 6 nucleotides in length and hybridizes under stringent conditions to the PEDF nucleic acid molecule (i.e., the nucleotide sequence of SEQ ID NO: 2 or SEQ ID NO:4). In another embodiment, the nucleic acid is at least 10, 25, 50, 100, 250, 500, 1000, 1500, 2000, or more nucleotides in length. In another embodiment, an isolated nucleic acid molecule hybridizes to the coding region, for example SEQ ID NO: 2 or SEQ ID NO:4.

As used herein, the term “hybridizes under stringent conditions” is intended to describe conditions for hybridization and washing under which nucleotide sequences at least 60% homologous to each other typically remain hybridized to each other. Moreover, as used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel et al., (eds.), Current Protocols in Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known in the art. See, e.g., Ausubel et al. (eds.), 1993, Current Protocols in Molecular Biology, John Wiley & Sons, N Y, and Kriegler, 1990, Gene Transfer and Expression, A Laboratory Manual, Stockton Press, NY. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel et al. (eds.), 1993, Current Protocols in Molecular Biology, John Wiley & Sons, N Y, and Kriegler, 1990, Gene Transfer and Expression, A Laboratory Manual, Stockton Press, NY; Shilo and Weinberg, 1981, Proc Natl Acad Sci USA 78: 6789-6792.

Also provided are PEDF polypeptides encoded by any of the nucleic acid molecules described herein. The invention also involves an isolated polypeptide that is at least 80% identical to a polypeptide having an amino acid sequence of SEQ ID NO:1. Alternatively, the isolated polypeptide is at least 80% homologous to a fragment (i.e., at least 6 contiguous amino acids) of a polypeptide having an amino acid sequence of SEQ ID NO: 1. Moreover, the invention also includes isolated polypeptides that are at least 80% homologous to a derivative, analog, or homolog of a polypeptide having an amino acid sequence of SEQ ID NO:1. Similarly, the invention also provides an isolated polypeptide that is at least 80% identical to a naturally occurring allelic variant of a polypeptide having an amino acid sequence of SEQ ID NO:1. Those skilled in the art will recognize that such polypeptides should be encoded by a nucleic acid molecule capable of hybridizing to a nucleic acid molecule of SEQ ID NO:2 or SEQ ID NO:4 under stringent conditions.

As used herein, the terms “protein” and “polypeptide” and the like are intended to be interchangeable. The polypeptides include PEDF polypeptides whose sequence is provided in SEQ ID NO:1. The invention also includes mutant or variant polypeptides any of whose residues may be changed from the corresponding residue shown in SEQ ID NO:1, while still encoding a polypeptide that maintains its PEDF activities and physiological functions, or a functional fragment thereof. In the mutant or variant protein, up to 20% or more of the residues may be so changed.

In general, a PEDF variant that preserves PEDF-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution.

Those skilled in the art will recognize that the invention also pertains to isolated PEDF polypeptides, and biologically active portions thereof, or derivatives, fragments, analogs or homologs thereof. PEDF constructs described herein can be isolated from cells or tissue sources by an appropriate purification scheme using standard protein purification techniques. In another embodiment, the PEDF polypeptides are produced by recombinant DNA techniques. As an alternative to recombinant expression, a PEDF protein or polypeptide can be synthesized chemically using standard peptide synthesis techniques.

An “isolated” or “purified” polypeptide or biologically active portion thereof is substantially free of cellular material or other contaminating proteins or polypeptides from the cell or tissue source from which the PEDF polypeptide is derived, or substantially free from chemical precursors or other chemicals when chemically synthesized. The language “substantially free of cellular material” includes preparations of PEDF polypeptides in which the polypeptide is separated from cellular components of the cells from which it is isolated or recombinantly produced. For example, the language “substantially free of cellular material” includes preparations of PEDF polypeptide having less than about 30% (by dry weight) of non-PEDF protein (also referred to herein as a “contaminating protein”), more preferably less than about 20% of non-PEDF protein, still more preferably less than about 10% of non-PEDF protein, and most preferably less than about 5% non-PEDF protein. When the PEDF polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation.

Similarly, the language “substantially free of chemical precursors or other chemicals” includes preparations of PEDF polypeptide in which the polypeptide is separated from chemical precursors or other chemicals that are involved in the synthesis of the polypeptide. For example, the language “substantially free of chemical precursors or other chemicals” includes preparations of PEDF polypeptide having less than about 30% (by dry weight) of chemical precursors or non-PEDF chemical, more preferably less than about 20% chemical precursors or non-PEDF chemicals, still more preferably less than about 10% chemical precursors or non-PEDF chemicals, and most preferably less than about 5% chemical precursors or non-PEDF chemicals.

Biologically active portions of a PEDF polypeptide construct include peptides comprising amino acid sequences sufficiently homologous to or derived from the amino acid sequence of the PEDF polypeptides, e.g., the amino acid sequence shown in SEQ ID NO:1, that include fewer amino acids than the full length PEDF constructs described herein, and exhibit at least one activity of a PEDF polypeptide. Typically, biologically active portions comprise a domain or motif with at least one activity of the PEDF polypeptide.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”). The nucleic acid sequence homology may be determined as the degree of identity between two sequences. The homology may be determined using computer programs known in the art, such as GAP software provided in the GCG program package. See, Needleman and Wunsch 1970 J Mol Biol 48: 443-453. The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

The invention further provides vectors containing any of the PEDF nucleic acid molecules. Specifically, the invention also pertains to vectors, preferably expression vectors, containing a nucleic acid encoding the PEDF polypeptides, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

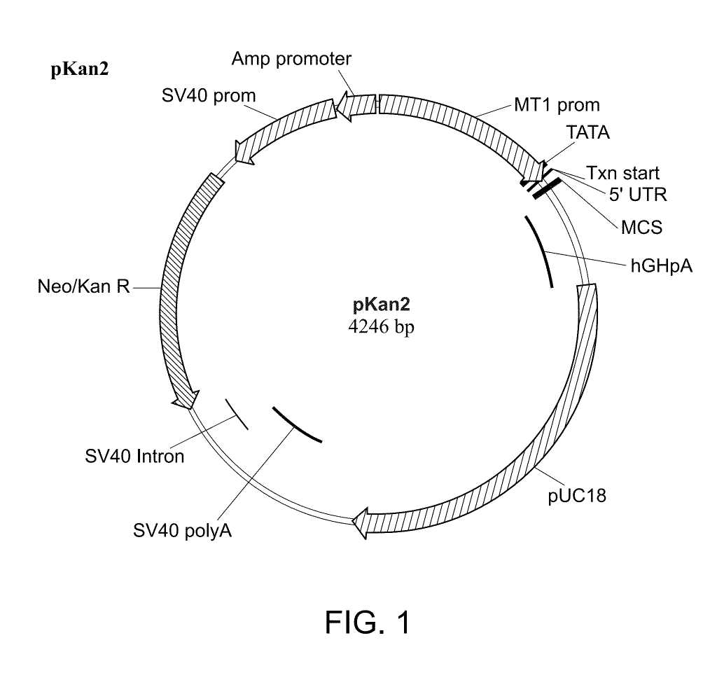

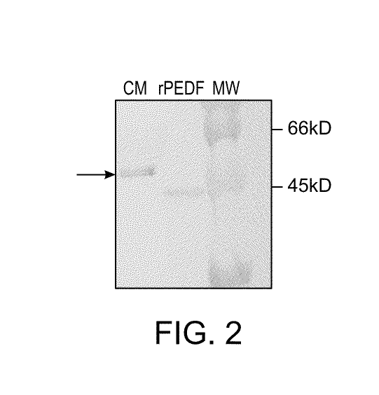

In one embodiment, the polynucleotide encoding PEDF is a recombinant construct such as a plasmid expression vector under the operative control of regulatory elements such as promoters, enhancers, secretory signals, termination signals, and the like. Methods for constructing suitable expression vectors are known in the art and are described, for example, in Sambrook et al., 1989, Molecular Cloning: A Laboratory Manual (2nd Ed.), Cold Spring Harbor Press, N.Y, and similar texts. Additionally, expression vectors are also commercially available. One preferred expression vector is the pKan2 vector (Neurotech) (see FIG. 1). To create a pKanX vector (i.e., pKan2 or other versions, where X=version), the pNUT expression vector, which has previously been used for the delivery of CNTF, was extensively modified. Recombinant techniques were used to make the following modifications to pNUT: 1) Ampicillin resistance gene (AmpR) deleted; 2) DHFR and HSV1 Thymidine Kinase cassettes deleted; 3) AmpR promoter place upstream of neomycin/kanamycin resistance gene (NeoR/KanR) to express kanamycin resistance gene in prokaryotes.

The recombinant expression vectors comprise any of PEDF nucleic acids in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors can be introduced into host cells to thereby produce proteins or peptides, including fusion proteins or peptides, encoded by nucleic acids as described herein (e.g., PEDF polypeptides, mutant forms of PEDF polypeptides, fusion proteins, etc.).

The recombinant expression vectors can be designed for expression of PEDF constructs in prokaryotic or eukaryotic cells. Other suitable expression systems for both prokaryotic and eukaryotic cells are known in the art. (See, e.g., Chapters 16 and 17 of Sambrook et al., Molecular Cloning: A Laboratory Manual. 2nd ed., Cold Spring Harbor Laboratory, Cold Spring Harbor Laboratory Press, Cold Spring Harbor, N. Y., 1989). A wide variety of host/expression vector combinations may be used to express the gene encoding the growth factor, or other biologically active molecule(s) of interest. Long-term, stable in vivo expression is achieved using expression vectors (i.e., recombinant DNA molecules) in which the gene encoding PEDF is operatively linked to a promoter that is not subject to down regulation upon implantation in-vivo in a mammalian host. Suitable promoters include, for example, strong constitutive mammalian promoters, such as beta-actin, eIF4A1, GAPDH, etc. Stress-inducible promoters, such as the metallothionein 1 (MT-1) or VEGF promoter may also be suitable. Additionally, hybrid promoters containing a core promoter and custom 5′ UTR or enhancer elements may be used. Other known non-retroviral promoters capable of controlling gene expression, such as CMV or the early and late promoters of SV40 or adenovirus are suitable.

The expression vector containing the gene of interest may then be used to transfect the desired cell line. Standard transfection techniques such as liposomal, calcium phosphate co-precipitation, DEAE-dextran transfection or electroporation may be utilized. Commercially available mammalian transfection kits, such as Fugene6 (Roche Applied Sciences), may be purchased. Human mammalian cells can be used. In all cases, it is important that the cells or tissue contained in the device are not contaminated or adulterated.

Preferred promoters used in the disclosed constructs include the SV40 promoter, the Amp promoter and/or the MT1 promoter.

Other useful expression vectors, for example, may consist of segments of chromosomal, non-chromosomal and synthetic DNA sequences, such as various known derivatives of SV40 and known bacterial plasmids, e.g., pUC, pBlueScript™ plasmids from E. coli including pBR322, pCR1, pMB9 and their derivatives. Expression vectors containing the geneticin (G418) or hygromycin drug selection genes (see Southern, P. J., In vitro, 18:315 (1981) and Southern, P. J. et al., Mol. Appl. Genet., 1:327 (1982)) are also useful. These vectors can employ a variety of different enhancer/promoter regions to drive the expression of both a biologic gene of interest and/or a gene conferring resistance to selection with toxin such as G418 or hygromycin B. A variety of different mammalian promoters can be employed to direct the expression of the genes for G418 and hygromycin B and/or the biologic gene of interest. The G418 resistance gene codes for aminoglycoside phosphotransferase (APH) which enzymatically inactivates G418 (100-1000 μg/μl) added to the culture medium. Only those cells expressing the APH gene will survive drug selection usually resulting in the expression of the second biologic gene as well. The hygromycin B phosphotransferase (HPH) gene codes for an enzyme which specifically modifies hygromycin toxin and inactivates it. Genes co-transfected with or contained on the same plasmid as the hygromycin B phosphotransferase gene will be preferentially expressed in the presence of hygromycin B at 50-200 μg/ml concentrations.

Examples of expression vectors that can be employed include, but are not limited to, the commercially available pRC/CMV, pRC/RSV, and pCDNAlNEO (InVitrogen).

In one embodiment, the pNUT expression vector, which contains the cDNA of the mutant DHFR and the entire pUC18 sequence including the polylinker, can be used. See, e.g., Aebischer, P., et al., Transplantation, 58, pp. 1275-1277 (1994); Baetge et al., PNAS, 83, pp. 5454-58 (1986). The pNUT expression vector can be modified such that the DHFR coding sequence is replaced by the coding sequence for G418 or hygromycin drug resistance. The SV40 promoter within the pNUT expression vector can also be replaced with any suitable constitutively expressed mammalian promoter, such as those discussed above. The genes encoding PEDF has been cloned and their nucleotide sequences published. (see GenBank Accession P36955). Other genes encoding the biologically active molecules useful in this invention that are not publicly available may be obtained using standard recombinant DNA methods such as PCR amplification, genomic and cDNA library screening with oligonucleotide probes. Any of the known genes coding for biologically active molecules may be employed in the methods and devices of this invention.

In addition, the invention also provides host cells or cell lines containing such vectors (or any of the nucleic acid molecules described herein). As used herein, the terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. By way of non-limiting example, the host cell may be an ARPE-19 cell. However, other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd ed., Cold Spring Harbor Laboratory, Cold Spring Harbor Laboratory Press, Cold Spring Harbor, N. Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the PEDF construct or can be introduced on a separate vector. Cells stably transfected with the introduced nucleic acid can be identified by drug selection (e.g., cells that have incorporated the selectable marker gene will survive, while the other cells die).

A host cell, such as a prokaryotic or eukaryotic host cell in culture, can be used to produce (i.e., express) a PEDF construct. Accordingly, the invention further provides methods for producing the PEDF polypeptides using host cells. In one embodiment, the method comprises culturing the host cell of invention (into which a recombinant expression vector encoding PEDF has been introduced) in a suitable medium such that PEDF polypeptide is produced. In another embodiment, the method further comprises isolating PEDF from the medium or the host cell.

Likewise, the invention also provides cell lines of ARPE-19 cells genetically engineered to produce PEDF, wherein, for example, the PEDF is encoded by a nucleic acid sequence of SEQ ID NO:2 or SEQ ID NO:4. Similarly, the invention also provides cell lines of ARPE-19 cells genetically engineered to produce PEDF comprising an amino acid sequence selected of SEQ ID NO:1.

To be a platform cell line for an encapsulated cell based delivery system, the cell line should have as many of the following characteristics as possible: (1) the cells should be hardy under stringent conditions (the encapsulated cells should be functional in the avascular tissue cavities such as in the central nervous system or the eye, especially in the intra-ocular environment); (2) the cells should be able to be genetically modified (the desired therapeutic factors needed to be engineered into the cells); (3) the cells should have a relatively long life span (the cells should produce sufficient progenies to be banked, characterized, engineered, safety tested and clinical lot manufactured); (4) the cells should preferably be of human origin (which increases compatibility between the encapsulated cells and the host); (5) the cells should exhibit greater than 80% viability for a period of more than one month in vivo in device (which ensures long-term delivery); (6) the encapsulated cells should deliver an efficacious quantity of a useful biological product (which ensures effectiveness of the treatment); (7) the cells should have a low level of host immune reaction (which ensures the longevity of the graft); and (8) the cells should be nontumorgenic (to provide added safety to the host, in case of device leakage).

Preferably, the cells for use according to the present invention are normal retinal pigmented epithelial. In a specific embodiment, the cells are ARPE-19 cells, which demonstrate all of the characteristics of a successful platform cell for an encapsulated cell-based delivery system (Dunn et al., Exp. Eye Res. (1996) 62:155-169; Dunn et al., Invest. Ophthalmol. Vis. Sci. (1998) 39:2744-9; Finnemann et al., Proc. Natl. Acad. Sci. U.S.A. (1997) 94:12932-12937; Handa et al., Exp. Eye (1998) 66:411-419; Holtkamp et al., Clin. Exp. Immunol. (1998) 112:34-43; and Maidji et al., J. Virol. (1996) 70:8402-8410). The use of ARPE-19 cells for encapsulated cell-based delivery of therapeutic agents is described in U.S. Pat. No. 6,361,771. ARPE-19 cells are available from the American Type Culture Collection (ATCC Number CRL-2302). ARPE-19 cells are normal retinal pigmented epithelial (RPE) cells and express the retinal pigmentary epithelial cell-specific markers CRALBP and RPE-65. ARPE-19 cells form stable monolayers, which exhibit morphological and functional polarity.

When the devices of the invention are used, preferably between 10² and 10⁸ engineered ARPE-19 cells, most preferably 5×10² to 5×10⁵ ARPE-19 cells that have been genetically engineered to secrete PEDF are encapsulated in each device. Dosage may be controlled by implanting a fewer or greater number of capsules, preferably between 1 and 50 capsules per patient. The devices described herein are capable of delivering between about 1.0 ng and 1000 ng of PEDF per eye per patient per day.

Techniques and procedures for isolating cells or tissues which produce a selected product are known to those skilled in the art, or can be adapted from known procedures with no more than routine experimentation.

If the cells to be isolated are replicating cells or cell lines adapted to growth in vitro, it is particularly advantageous to generate a cell bank of these cells. A particular advantage of a cell bank is that it is a source of cells prepared from the same culture or batch of cells. That is, all cells originated from the same source of cells and have been exposed to the same conditions and stresses. Therefore, the vials can be treated as homogenous culture. In the transplantation context, this greatly facilitates the production of identical or replacement devices. It also allows simplified testing protocols, which assure that implanted cells are free of retroviruses and the like. It may also allow for parallel monitoring of vehicles in vivo and in vitro, thus allowing investigation of effects or factors unique to residence in vivo.

The instant invention also relates to biocompatible, optionally immunoisolatory, devices for the delivery PEDF to the eye. Such devices contain a core containing living cells that produce or secrete PEDF and a biocompatible jacket surrounding the core, wherein the jacket has a molecular weight cut off (“MWCO”) that allows the diffusion of PEDF into the eye and to the central nervous system, including the brain, ventricle, spinal cord.

A variety of biocompatible capsules are suitable for delivery of molecules according to this invention. Useful biocompatible polymer capsules comprise (a) a core which contains a cell or cells, either suspended in a liquid medium or immobilized within a biocompatible matrix, and (b) a surrounding jacket comprising a membrane which does not contain isolated cells, which is biocompatible, and permits diffusion of the cell-produced biologically active molecule into the eye.

Many transformed cells or cell lines are advantageously isolated within a capsule having a liquid core, comprising, e.g., a nutrient medium, and optionally containing a source of additional factors to sustain cell viability and function. The core of the devices of the invention can function as a reservoir for growth factors (e.g., prolactin, or insulin-like growth factor 2), growth regulatory substances such as transforming growth factor β (TGF-β) or the retinoblastoma gene protein or nutrient-transport enhancers (e.g., perfluorocarbons, which can enhance the concentration of dissolved oxygen in the core). Certain of these substances are also appropriate for inclusion in liquid media.

In addition, the instant devices can also be used as a reservoir for the controlled delivery of needed drugs or biotherapeutics. In such cases, the core contains a high concentration of the selected drug or biotherapeutic (alone or in combination with cells or tissues). Moreover, satellite vehicles containing substances which prepare or create a hospitable environment in the area of the body in which a device according to the invention is implanted can also be implanted into a recipient. In such instances, the devices containing immunoisolated cells are implanted in the region along with satellite vehicles releasing controlled amounts of, for example, a substance which down-modulates or inhibits an inflammatory response from the recipient (e.g., anti-inflammatory steroids), or a substance which stimulates the ingrowth of capillary beds (e.g., an angiogenic factor).

Alternatively, the core may comprise a biocompatible matrix of a hydrogel or other biocompatible, three-dimensional material (e.g., extracellular matrix components) which stabilizes the position of the cells. The term “hydrogel” herein refers to a three dimensional network of cross-linked hydrophilic polymers. The network is in the form of a gel, substantially composed of water, preferably gels being greater than 90% water. Compositions which form hydrogels fall into three classes. The first class carries a net negative charge (e.g., alginate). The second class carries a net positive charge (e.g., collagen and laminin). Examples of commercially available extracellular matrix components include Matrigel™ and Vitrogen™. The third class is net neutral in charge (e.g., highly crosslinked polyethylene oxide, or polyvinylalcohol).

Any suitable matrix or spacer may be employed within the core, including precipitated chitosan, synthetic polymers and polymer blends, microcarriers and the like, depending upon the growth characteristics of the cells to be encapsulated.

Alternatively, the capsule may have an internal scaffold. The scaffold may prevent cells from aggregating and improve cellular distribution within the device. (See PCT publication no. WO 96/02646). The scaffold defines the microenvironment for the encapsulated cells and keeps the cells well distributed within the core. The optimal internal scaffold for a particular device is highly dependent on the cell type to be used. In the absence of such a scaffold, adherent cells aggregate to form clusters.

For example, the internal scaffold may be a yarn or a mesh. The filaments used to form a yarn or mesh internal scaffold are formed of any suitable biocompatible, substantially non-degradable material. (See U.S. Pat. Nos. 6,303,136 and 6,627,422, which are herein incorporated by reference). Preferably, the capsule of this invention will be similar to those described by PCT International patent applications WO 92/19195 or WO 95/05452, incorporated by reference; or U.S. Pat. Nos. 5,639,275; 5,653,975; 4,892,538; 5,156,844; 5,283,187; or 5,550,050, incorporated by reference. Materials useful in forming yarns or woven meshes include any biocompatible polymers that are able to be formed into fibers such as, for example, acrylic, polyester, polyethylene, polypropylene, polyacrylonitrile, polyethylene terephthalate, nylon, polyamides, polyurethanes, polybutester, or natural fibers such as cotton, silk, chitin or carbon. Any suitable thermoplastic polymer, thermoplastic elastomer, or other synthetic or natural material having fiber-forming properties may be inserted into a pre-fabricated hollow fiber membrane or a hollow cylinder formed from a flat membrane sheet. For example, silk, PET or nylon filaments used for suture materials or in the manufacture of vascular grafts are highly conducive to this type of application. In other embodiments, metal ribbon or wire may be used and woven. Each of these filament materials has well-controlled surface and geometric properties, may be mass produced, and has a long history of implant use. In certain embodiments, the filaments may be “texturized” to provide rough surfaces and “hand-holds” onto which cell projections may attach. The filaments may be coated with extracellular matrix molecules or surface-treated (e.g. plasma irradiation) to enhance cellular adhesion to the filaments.

In some embodiments, the filaments, preferably organized in a non-random unidirectional orientation, are twisted in bundles to form yarns of varying thickness and void volume. Void volume is defined as the spaces existing between filaments. The void volume in the yarn should vary between 20-95%, but is preferably between 50-95%. The preferred void space between the filaments is between 20-200 μm, sufficient to allow the scaffold to be seeded with cells along the length of the yarn, and to allow the cells to attach to the filaments. The preferred diameter of the filaments comprising the yarn is between 5-100 μm. These filaments should have sufficient mechanical strength to allow twisting into a bundle to comprise a yarn. The filament cross-sectional shape can vary, with circular, rectangular, elliptical, triangular, and star-shaped cross-section being preferred.

Alternatively, the filaments or yarns can be woven into a mesh. The mesh can be produced on a braider using carriers, similar to bobbins, containing monofilaments or multifilaments, which serve to feed either the yarn or filaments into the mesh during weaving. The number of carriers is adjustable and may be wound with the same filaments or a combination of filaments with different compositions and structures. The angle of the braid, defined by the pick count, is controlled by the rotational speed of the carriers and the production speed. In one embodiment, a mandrel is used to produce a hollow tube of mesh. In certain embodiments, the braid is constructed as a single layer, in other embodiments it is a multi-layered structure. The tensile strength of the braid is the linear summation of the tensile strengths of the individual filaments.

In other embodiments, a tubular braid is constructed. The braid can be inserted into a hollow fiber membrane upon which the cells are seeded. Alternatively, the cells can be allowed to infiltrate the wall of the mesh tube to maximize the surface area available for cell attachment. When such cell infiltration occurs, the braid serves both as a cell scaffold matrix and as an inner support for the device. The increase in tensile strength for the braid-supported device is significantly higher than in alternative approaches.

As noted, for implant sites that are not immunologically privileged, such as periocular sites, and other areas outside the anterior chamber (aqueous) and the posterior chamber (vitreous), the capsules are preferably immunoisolatory. Components of the biocompatible material may include a surrounding semipermeable membrane and the internal cell-supporting scaffolding. The transformed cells are preferably seeded onto the scaffolding, which is encapsulated by the permselective membrane, which is described above. Also, bonded fiber structures can be used for cell implantation. (See U.S. Pat. No. 5,512,600, incorporated by reference). Biodegradable polymers include those comprised of poly(lactic acid) PLA, poly(lactic-coglycolic acid) PLGA, and poly(glycolic acid) PGA and their equivalents. Foam scaffolds have been used to provide surfaces onto which transplanted cells may adhere (PCT International patent application Ser. No. 98/05304, incorporated by reference). Woven mesh tubes have been used as vascular grafts (PCT International patent application WO 99/52573, incorporated by reference). Additionally, the core can be composed of an immobilizing matrix formed from a hydrogel or other biocompatible three-dimensional matrix, which stabilizes the position of the cells. A hydrogel is a 3-dimensional network of cross-linked hydrophilic polymers in the form of a gel, substantially composed of water.

Various polymers and polymer blends can be used to manufacture the surrounding semipermeable membrane, including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Preferably, the surrounding semipermeable membrane is a biocompatible semipermeable hollow fiber membrane. Such membranes, and methods of making them are disclosed by U.S. Pat. Nos. 5,284,761 and 5,158,881, incorporated by reference. The surrounding semipermeable membrane is formed from a polyether sulfone hollow fiber, such as those described by U.S. Pat. No. 4,976,859 or U.S. Pat. No. 4,968,733, incorporated by reference. An alternate surrounding semipermeable membrane material is polysulfone.

The capsule can be any configuration appropriate for maintaining biological activity and providing access for delivery of the product or function, including for example, cylindrical, rectangular, disk-shaped, patch-shaped, ovoid, stellate, or spherical. Moreover, the capsule can be coiled or wrapped into a mesh-like or nested structure. If the capsule is to be retrieved after it is implanted, configurations which tend to lead to migration of the capsules from the site of implantation, such as spherical capsules small enough to travel in the recipient host's blood vessels, are not preferred. Certain shapes, such as rectangles, patches, disks, cylinders, and flat sheets offer greater structural integrity and are preferable where retrieval is desired.

Preferably, the device has a tether that aids in maintaining device placement during implant, and aids in retrieval. Such a tether may have any suitable shape that is adapted to secure the capsule in place. For example, the suture may be a loop, a disk, or a suture. In some embodiments, the tether is shaped like an eyelet, so that suture may be used to secure the tether (and thus the device) to the sclera, or other suitable ocular structure. In another embodiment, the tether is continuous with the capsule at one end, and forms a pre-threaded suture needle at the other end. In one preferred embodiment, the tether is an anchor loop that is adapted for anchoring the capsule to an ocular structure. The tether may be constructed of a shape memory metal and/or any other suitable medical grade material known in the art.

In a hollow fiber configuration, the fiber will have an inside diameter of less than 1000 microns, preferably less than 750 microns. Devices having an outside diameter less than 300-600 microns are also contemplated. For implantation in the eye, in a hollow fiber configuration the capsule will preferably be between 0.4 cm to 1.5 cm in length, most preferably between 0.4 to 1.0 cm in length. Longer devices may be accommodated in the eye, however, a curved or arcuate shape may be required for secure and appropriate placement. The hollow fiber configuration is preferred for intraocular placement.

For periocular placement, either a hollow fiber configuration (with dimensions substantially as above) or a flat sheet configuration is contemplated. The upper limit contemplated for a flat sheet is approximately 5 mm×5 mm—assuming a square shape. Other shapes with approximately the same surface area are also contemplated.

The hydraulic permeability will typically be in the range of 1-100 mls/min/m²/mmHg., for example, 0.5-100 mls/min/m²/mmHg, preferably in the range of 1-70 mls/min/m²/mmHg. The glucose mass transfer coefficient of the capsule can be defined, measured, and calculated as described by Dionne et al., ASAIO, Abstracts, p. 99 (1993) and Colton et al., The Kidney, eds. Brenner B M and Rector F C, pp. 2425-89 (1981) (both of which are incorporated herein by reference in their entireties.

The surrounding or peripheral region (jacket), which surrounds the core of the instant devices can be permselective, biocompatible, and/or immunoisolatory. It is produced in such a manner that it is free of isolated cells, and completely surrounds (i.e., isolates) the core, thereby preventing contact between any cells in the core and the recipient's body. Biocompatible semi-permeable hollow fiber membranes, and methods of making them are disclosed in U.S. Pat. Nos. 5,284,761 and 5,158,881 (See also, WO 95/05452), each of which incorporated herein by reference in its entirety. For example, the capsule jacket can be formed from a polyether sulfone hollow fiber, such as those described in U.S. Pat. Nos. 4,976,859 and 4,968,733, and 5,762,798, each incorporated herein by reference.

To be permselective, the jacket is formed in such a manner that it has a molecular weight cut off (“MWCO”) range appropriate both to the type and extent of immunological reaction anticipated to be encountered after the device is implanted and to the molecular size of the largest substance whose passage into and out of the device into the eye is desirable. The type and extent of immunological attacks which may be mounted by the recipient following implantation of the device depend in part upon the type(s) of moiety isolated within it and in part upon the identity of the recipient (i.e., how closely the recipient is genetically related to the source of the BAM). When the implanted tissue or cells are allogeneic to the recipient, immunological rejection may proceed largely through cell-mediated attack by the recipient's immune cells against the implanted cells. When the tissue or cells are xenogeneic to the recipient, molecular attack through assembly of the recipient's cytolytic complement attack complex may predominate, as well as the antibody interaction with complement.

The jacket allows passage into the eye of substances up to a predetermined size, but prevents the passage of larger substances. More specifically, the surrounding or peripheral region is produced in such a manner that it has pores or voids of a predetermined range of sizes, and, as a result, the device is permselective. The MWCO of the surrounding jacket must be sufficiently low to prevent access of the substances required to carry out immunological attacks to the core, yet sufficiently high to allow delivery of PEDF to the recipient's eye. Preferably, when PEDF is used, the MWCO of the biocompatible jacket of the devices of the instant invention is from about 1 kD to about 1500 kD (e.g., from about 50 to about 1500 kD). However an open membrane with a MWCO greater than 200 kD can also be used.

As used herein with respect to the jacket of the device, the term “biocompatible” refers collectively to both the device and its contents. Specifically, it refers to the capability of the implanted intact device and its contents to avoid the detrimental effects of the body's various protective systems and to remain functional for a significant period of time. As used herein, the term “protective systems” refers to the types of immunological attack which can be mounted by the immune system of an individual in whom the instant vehicle is implanted, and to other rejection mechanisms, such as the fibrotic response, foreign body response and other types of inflammatory response which can be induced by the presence of a foreign object in the individuals' body. In addition to the avoidance of protective responses from the immune system or foreign body fibrotic response, the term “biocompatible”, as used herein, also implies that no specific undesirable cytotoxic or systemic effects are caused by the vehicle and its contents such as those that would interfere with the desired functioning of the vehicle or its contents.

The external surface of the device can be selected or designed in such a manner that it is particularly suitable for implantation at a selected site. For example, the external surface can be smooth, stippled or rough, depending on whether attachment by cells of the surrounding tissue is desirable. The shape or configuration can also be selected or designed to be particularly appropriate for the implantation site chosen.

The biocompatibility of the surrounding or peripheral region (jacket) of the device is produced by a combination of factors. Important for biocompatibility and continued functionality are device morphology, hydrophobicity and the absence of undesirable substances either on the surface of, or leachable from, the device itself. For example, if a charge modification is made to the membrane which allows the increased passage of positively charged molecules, the modified membrane will most likely be hydrophobic. Thus, brush surfaces, folds, interlayers or other shapes or structures eliciting a foreign body response are avoided. Moreover, the device-forming materials are sufficiently pure to insure that unwanted substances do not leach out from the device materials themselves. Additionally, following device preparation, the treatment of the external surface of the device with fluids or materials (e.g. serum) which may adhere to or be absorbed by the device and subsequently impair device biocompatibility is avoided.

First, the materials used to form the device jacket are substances selected based upon their ability to be compatible with, and accepted by, the tissues of the recipient of the implanted device. Substances are used which are not harmful to the recipient or to the isolated cells. Preferred substances include polymer materials, i.e., thermoplastic polymers. Particularly preferred thermoplastic polymer substances are those which are modestly hydrophobic, i.e. those having a solubility parameter as defined in Brandrup J., et al. Polymer Handbook 3rd Ed., John Wiley & Sons, NY (1989), between 8 and 15, or more preferably, between 9 and 14 (Joules/m³)^{1/2}. The polymer substances are chosen to have a solubility parameter low enough so that they are soluble in organic solvents and still high enough so that they will partition to form a proper membrane. Such polymer substances should be substantially free of labile nucleophilic moieties and be highly resistant to oxidants and enzymes even in the absence of stabilizing agents. The period of residence in vivo which is contemplated for the particular vehicle must also be considered: substances must be chosen which are adequately stable when exposed to physiological conditions and stresses. Many thermoplastics are known which are sufficiently stable, even for extended periods of residence in vivo, such as periods in excess of one or two years.

The choice of materials used to construct the device is determined by a number of factors as described in detail in Dionne WO 92/19195, herein incorporated by reference. Briefly, various polymers and polymer blends can be used to manufacture the capsule jacket. Polymeric membranes forming the device and the growth surfaces therein may include polyacrylates (including acrylic copolymers), polyvinylidenes, polyvinyl chloride copolymers, polyurethanes, polystyrenes, polyamides, polymethylmethacrylate, polyvinyldifluoride, polyolefins, cellulose acetates, cellulose nitrates, polysulfones, polyphosphazenes, polyacrylonitriles, poly(acrylonitrile/covinyl chloride), as well as derivatives, copolymers and mixtures thereof.

A preferred membrane casting solution comprises a either polysulfone dissolved in the water-miscible solvent dimethylacetamide (DMACSO) or polyethersulfone dissolved in the water-miscible solvent butyrolactone. This casting solution can optionally comprise hydrophilic or hydrophobic additives which affect the permeability characteristics of the finished membrane. A preferred hydrophilic additive for the polysulfone or polyethersulfone is polyvinylpyrrolidone (PVP). Other suitable polymers comprise polyacrylonitrile (PAN), polymethylmethacrylate (PMMA), polyvinyldifluoride (PVDF), polyethylene oxide, polyolefins (e.g., polyisobutylene or polypropylene), polyacrylonitrile/polyvinyl chloride (PAN/PVC), and/or cellulose derivatives (e.g., cellulose acetate or cellulose butyrate). Compatible water-miscible solvents for these and other suitable polymers and copolymers are found in the teachings of U.S. Pat. No. 3,615,024.

Second, substances used in preparing the biocompatible jacket of the device are either free of leachable pyrogenic or otherwise harmful, irritating, or immunogenic substances or are exhaustively purified to remove such harmful substances. Thereafter, and throughout the manufacture and maintenance of the device prior to implantation, great care is taken to prevent the adulteration or contamination of the device or jacket with substances, which would adversely affect its biocompatibility.

Third, the exterior configuration of the device, including its texture, is formed in such a manner that it provides an optimal interface with the eye of the recipient after implantation. Certain device geometries have also been found to specifically elicit foreign body fibrotic responses and should be avoided. Thus, devices should not contain structures having interlayers such as brush surfaces or folds. In general, opposing vehicle surfaces or edges either from the same or adjacent vehicles should be at least 1 mm apart, preferably greater than 2 mm and most preferably greater than 5 mm. Preferred embodiments include cylinders having an outer diameter of between about 200 and 350 μm and a length between about 0.4 and 6 mm. Preferably, the core of the devices of the invention have a volume of approximately 2.5 However, those skilled in the art will recognize that it is also possible to use “micronized” devices having a core volume of less than 0.5 μl (e.g., about 0.3 μl).

The surrounding jacket of the biocompatible devices can optionally include substances which decrease or deter local inflammatory response to the implanted vehicle and/or generate or foster a suitable local environment for the implanted cells or tissues. For example antibodies to one or more mediators of the immune response could be included. Available potentially useful antibodies such as antibodies to the lymphokines tumor necrosis factor (TNF), and to interferons (IFN) can be included in the matrix precursor solution. Similarly, an anti-inflammatory steroid can be included. See Christenson, L., et al., J. Biomed. Mat. Res., 23, pp. 705-718 (1989); Christenson, L., Ph.D. thesis, Brown University, 1989, herein incorporated by reference. Alternatively, a substance which stimulates angiogenesis (ingrowth of capillary beds) can be included.

In some embodiments, the jacket of the present device is immunoisolatory. That is, it protects cells in the core of the device from the immune system of the individual in whom the device is implanted. It does so (1) by preventing harmful substances of the individual's body from entering the core, (2) by minimizing contact between the individual and inflammatory, antigenic, or otherwise harmful materials which may be present in the core and (3) by providing a spatial and physical barrier sufficient to prevent immunological contact between the isolated moiety and detrimental portions of the individual's immune system.

For example, the external jacket may be either an ultrafiltration membrane or a microporous membrane. Those skilled in the art will recognize that ultrafiltration membranes are those having a pore size range of from about 1 to about 100 nanometers while a microporous membrane has a range of between about 0.05 to about 10 microns.

The thickness of this physical barrier can vary, but it will always be sufficiently thick to prevent direct contact between the cells and/or substances on either side of the barrier. The thickness of this region generally ranges between 5 and 200 microns; thicknesses of 10 to 100 microns are preferred, and thickness of 20 to 50 or 20 to 75 microns are particularly preferred. Types of immunological attack which can be prevented or minimized by the use of the instant device include attack by macrophages, neutrophils, cellular immune responses (e.g. natural killer cells and antibody-dependent T cell-mediated cytoloysis (ADCC)), and humoral response (e.g. antibody-dependent complement mediated cytolysis).

The capsule jacket may be manufactured from various polymers and polymer blends including polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), as well as derivatives, copolymers and mixtures thereof. Capsules manufactured from such materials are described, e.g., in U.S. Pat. Nos. 5,284,761 and 5,158,881, incorporated herein by reference. Capsules formed from a polyether sulfone (PES) fiber, such as those described in U.S. Pat. Nos. 4,976,859 and 4,968,733, incorporated herein by reference, may also be used.

Depending on the outer surface morphology, capsules have been categorized as Type 1 (T1), Type 2 (T2), Type ½ (T½), or Type 4 (T4). Such membranes are described, e.g., in Lacy et al., “Maintenance Of Normoglycemia In Diabetic Mice By Subcutaneous Xenografts Of Encapsulated Islets”, Science, 254, pp. 1782-84 (1991), Dionne et al., WO 92/19195 and Baetge, WO 95/05452. A smooth outer surface morphology is preferred.

Those skilled in the art will recognize that capsule jackets with permselective, immunoisolatory membranes are preferable for sites that are not immunologically privileged. In contrast, microporous membranes or permselective membranes may be suitable for immunologically privileged sites. For implantation into immunologically privileged sites, capsules made from the PES or PS membranes are preferred.

Any suitable method of sealing the capsules know in the art may be used, including the employment of polymer adhesives and/or crimping, knotting and heat sealing. In addition, any suitable “dry” sealing method can also be used. In such methods, a substantially non-porous fitting is provided through which the cell-containing solution is introduced. Subsequent to filling, the capsule is sealed. Such methods are described in, e.g., U.S. Pat. Nos. 5,653,688; 5,713,887; 5,738,673; 6,653,687; 5,932,460; and 6,123,700, which are herein incorporated by reference.

According to the methods of this invention, other molecules may be co-delivered in addition to PEDF. For example, it may be preferable to deliver a trophic factor(s) with an anti-angiogenic factor and/or a neuroprotective or neurotrophic factor such as PEDF.

Co-delivery can be accomplished in a number of ways. First, cells may be transfected with separate constructs containing the genes encoding the described molecules. Second, cells may be transfected with a single construct containing two or more genes as well as the necessary control elements. Third, two or more separately engineered cell lines can be either co-encapsulated or more than one device can be implanted at the site of interest.

Multiple gene expression from a single transcript is preferred over expression from multiple transcription units. See, e.g., Macejak, Nature, 353, pp. 90-94 (1991); WO 94/24870; Mountford and Smith, Trends Genet., 11, pp. 179-84 (1995); Dirks et al., Gene, 128, pp. 247-49 (1993); Martinez-Salas et al., J. Virology, 67, pp. 3748-55 (1993) and Mountford et al., Proc. Natl. Acad. Sci. USA, 91, pp. 4303-07 (1994).

For some indications, it may be preferable to deliver BAMs to two different sites in the eye concurrently. For example, it may be desirable to deliver a neurotrophic factor to the vitreous to supply the neural retina (ganglion cells to the RPE) and to deliver an anti-angiogenic factor via the sub-Tenon's space to supply the choroidal vasculature. Those skilled in the art will recognize that PEDF is both a neurotrophic factor as well as an anti-angiogenic factor. Accordingly, PEDF can serve both purposes by concurrently implanting the capsules of the invention into two or more different sites in the eyes.

This invention also contemplates use of different cell types during the course of the treatment regime. For example, a patient may be implanted with a capsule device containing a first cell type (e.g., BHK cells). If after time, the patient develops an immune response to that cell type, the capsule can be retrieved, or explanted, and a second capsule can be implanted containing a second cell type (e.g., CHO cells). In this manner, continuous provision of the therapeutic molecule is possible, even if the patient develops an immune response to one of the encapsulated cell types.

The methods and devices of this invention are intended for use in a primate, preferably human host, recipient, patient, subject or individual. A number of different implantation sites are contemplated for the devices and methods of this invention. Suitable implantation sites include, but are not limited to, the aqueous and vitreous humors of the eye, the periocular space, the anterior chamber, and/or the Subtenon's capsule.

The type and extent of immunological response by the recipient to the implanted device will be influenced by the relationship of the recipient to the isolated cells within the core. For example, if core contains syngeneic cells, these will not cause a vigorous immunological reaction, unless the recipient suffers from an autoimmunity with respect to the particular cell or tissue type within the device. Syngeneic cells or tissue are rarely available. In many cases, allogeneic or xenogeneic cells or tissue (i.e., from donors of the same species as, or from a different species than, the prospective recipient) may be available. The use of immunoisolatory devices allows the implantation of allogeneic or xenogeneic cells or tissue, without a concomitant need to immunosuppress the recipient. Use of immunoisolatory capsules also allows the use of unmatched cells (allographs). Therefore, the instant device makes it possible to treat many more individuals than can be treated by conventional transplantation techniques.

The type and vigor of an immune response to xenografted tissue is expected to differ from the response encountered when syngeneic or allogeneic tissue is implanted into a recipient. This rejection may proceed primarily by cell-mediated, or by complement-mediated attack. The exclusion of IgG from the core of the vehicle is not the touchstone of immunoprotection, because in most cases IgG alone is insufficient to produce cytolysis of the target cells or tissues. Using immunoisolatory devices, it is possible to deliver needed high molecular weight products or to provide metabolic functions pertaining to high molecular weight substances, provided that critical substances necessary to the mediation of immunological attack are excluded from the immunoisolatory capsule. These substances may comprise the complement attack complex component Clq, or they may comprise phagocytic or cytotoxic cells. Use of immunoisolatory capsules provides a protective barrier between these harmful substances and the isolated cells.

While the devices of the present invention are macrocapsules, those skilled in the art will recognize that microcapsules such as, for example those described in Rha, Lim, and Sun may also be used. (See, Rha, C. K. et al., U.S. Pat. No. 4,744,933; Methods in Enzymology 137, pp. 575-579 (1988); U.S. Pat. No. 4,652,833; U.S. Pat. No. 4,409,331). In general, microcapsules differ from macrocapsules by (1) the complete exclusion of cells from the outer layer of the device, and (2) the thickness of the outer layer of the device. Typically, microcapsules have a volume on the order of 1 μl and contain fewer than 10⁴ cells. More specifically, microencapsulation encapsulates approximately 1-10 viable islets or 500 cells, generally, per capsule.

Capsules with a lower MWCO may be used to further prevent interaction of molecules of the patient's immune system with the encapsulated cells.

Any of the devices used in accordance with the methods described herein must provide, in at least one dimension, sufficiently close proximity of any isolated cells in the core to the surrounding eye tissues of the recipient in order to maintain the viability and function of the isolated cells. However, the diffusional limitations of the materials used to form the device do not in all cases solely prescribe its configurational limits. Certain additives can be used which alter or enhance the diffusional properties, or nutrient or oxygen transport properties, of the basic vehicle. For example, the internal medium of the core can be supplemented with oxygen-saturated perfluorocarbons, thus reducing the needs for immediate contact with blood-borne oxygen. This will allow isolated cells or tissues to remain viable while, for instance, a gradient of angiotensin is released from the vehicle into the surrounding tissues, stimulating ingrowth of capillaries. References and methods for use of perfluorocarbons are given by Faithful, N. S. Anaesthesia, 42, pp. 234-242 (1987) and NASA Tech Briefs MSC-21480, U.S. Govt. Printing Office, Washington, D.C. 20402, incorporated herein by reference. Alternatively for clonal cell lines such as PC12 cells, genetically engineered hemoglobin sequences may be introduced into the cell lines to produce superior oxygen storage. See NPO-17517 NASA Tech Briefs, 15, p. 54.

The thickness of the device jacket should be sufficient to prevent an immunoresponse by the patient to the presence of the devices. For that purpose, the devices preferably have a minimum thickness of 1 μm or more and are free of the cells.

Additionally, reinforcing structural elements can also be incorporated into the devices. For example, these structural elements can be made in such a fashion that they are impermeable and are appropriately configured to allow tethering or suturing of the device to the eye tissues of the recipient. In certain circumstances, these elements can act to securely seal the jacket (e.g., at the ends of the cylinder), thereby completing isolation of the core materials (e.g., a molded thermoplastic clip). In many embodiments, it is desirable that these structural elements should not occlude a significant area of the permselective jacket.

The device of the present invention is of a sufficient size and durability for complete retrieval after implantation. One preferred device of the present invention has a core of a volume of approximately 1-3 uL. The internal geometry of micronized devices has a volume of approximately 0.05-0.1 uL.

Along with PEDF, at least one additional BAM can be delivered from the device to the eye. For example, the at least one additional BAM can be provided from a cellular or a noncellular source. When the at least one additional BAM is provided from a noncellular source, the additional BAM(s) may be encapsulated in, dispersed within, or attached to one or more components of the cell system including, but not limited to: (a) sealant; (b) scaffold; (c) jacket membrane; (d) tether anchor; and/or (e) core media. In such embodiment, co-delivery of the BAM from a noncellular source may occur from the same device as the BAM from the cellular source.

Alternatively, two or more encapsulated cell systems can be used. For example, the least one additional biologically active molecule can be a nucleic acid, a nucleic acid fragment, a peptide, a polypeptide, a peptidomimetic, a carbohydrate, a lipid, an organic molecule, an inorganic molecule, a therapeutic agent, or any combinations thereof. Specifically, the therapeutic agents may be an anti-angiogenic drug, a steroidal and non-steroidal anti-inflammatory drug, an anti-mitotic drug, an anti-tumor drug, an anti-parasitic drug, an TOP reducer, a peptide drug, and/or any other biologically active molecule drugs approved for ophthalmologic use.

Suitable excipients include, but are not limited to, any non-degradable or biodegradable polymers, hydrogels, solubility enhancers, hydrophobic molecules, proteins, salts, or other complexing agents approved for formulations.

Non-cellular dosages can be varied by any suitable method known in the art such as varying the concentration of the therapeutic agent, and/or the number of devices per eye, and/or modifying the composition of the encapsulating excipient. Cellular dosage can be varied by changing (1) the number of cells per device, (2) the number of devices per eye, and/or (3) the level of BAM production per cell. Cellular production can be varied by changing, for example, the copy number of the gene for the BAM in the transduced cell, or the efficiency of the promoter driving expression of the BAM. Suitable dosages from non-cellular sources may range from about 1 pg to about 1000 ng per day.

The instant invention also relates to methods for making the macrocapsular devices described herein. Devices may be formed by any suitable method known in the art. (See, e.g., U.S. Pat. Nos. 6,361,771; 5,639,275; 5,653,975; 4,892,538; 5,156,844; 5,283,138; and 5,550,050, each of which is incorporated herein by reference).

Membranes used can also be tailored to control the diffusion of molecules, such as PEDF, based on their molecular weight. (See Lysaght et al., 56 J. Cell Biochem. 196 (1996), Colton, 14 Trends Biotechnol. 158 (1996)). Using encapsulation techniques, cells can be transplanted into a host without immune rejection, either with or without use of immunosuppressive drugs. The capsule can be made from a biocompatible material that, after implantation in a host, does not elicit a detrimental host response sufficient to result in the rejection of the capsule or to render it inoperable, for example through degradation. The biocompatible material is relatively impermeable to large molecules, such as components of the host's immune system, but is permeable to small molecules, such as insulin, growth factors, and nutrients, while allowing metabolic waste to be removed. A variety of biocompatible materials are suitable for delivery of growth factors by the composition of the invention. Numerous biocompatible materials are known, having various outer surface morphologies and other mechanical and structural characteristics.

If a device with a jacket of thermoplastic or polymer membrane is desired, the pore size range and distribution can be determined by varying the solids content of the solution of precursor material (the casting solution), the chemical composition of the water-miscible solvent, or optionally including a hydrophilic or hydrophobic additive to the casting solution, as taught by U.S. Pat. No. 3,615,024. The pore size may also be adjusted by varying the hydrophobicity of the coagulant and/or of the bath.

Typically, the casting solution will comprise a polar organic solvent containing a dissolved, water-insoluble polymer or copolymer. This polymer or copolymer precipitates upon contact with a solvent-miscible aqueous phase, forming a permselective membrane at the site of interface. The size of pores in the membrane depends upon the rate of diffusion of the aqueous phase into the solvent phase; the hydrophilic or hydrophobic additives affect pore size by altering this rate of diffusion. As the aqueous phase diffuses farther into the solvent, the remainder of the polymer or copolymer is precipitated to form a trabecular support which confers mechanical strength to the finished device.

The external surface of the device is similarly determined by the conditions under which the dissolved polymer or copolymer is precipitated (i.e., exposed to the air, which generates an open, trabecular or sponge-like outer skin, immersed in an aqueous precipitation bath, which results in a smooth permselective membrane bilayer, or exposed to air saturated with water vapor, which results in an intermediate structure).

The surface texture of the device is dependent in part on whether the extrusion nozzle is positioned above, or immersed in, the bath: if the nozzle is placed above the surface of the bath a roughened outer skin will be formed, whereas if the nozzle is immersed in the bath a smooth external surface is formed.

The surrounding or peripheral matrix or membrane can be preformed, filled with the materials which will form the core (for instance, using a syringe), and subsequently sealed in such a manner that the core materials are completely enclosed. The device can then be exposed to conditions which bring about the formation of a core matrix if a matrix precursor material is present in the core.

The devices of the invention can provide for the implantation of diverse cell or tissue types, including fully-differentiated, anchorage-dependent, fetal or neonatal, or transformed, anchorage-independent cells or tissue. The cells to be isolated are prepared either from a donor (i.e., primary cells or tissues, including adult, neonatal, and fetal cells or tissues) or from cells which replicate in vitro (i.e., immortalized cells or cell lines, including genetically modified cells). In all cases, a sufficient quantity of cells to produce effective levels of the needed product or to supply an effective level of the needed metabolic function is prepared, generally under sterile conditions, and maintained appropriately (e.g. in a balanced salt solution such as Hank's salts, or in a nutrient medium, such as Ham's F12) prior to isolation.

The ECT devices of the invention are of a shape which tends to reduce the distance between the center of the device and the nearest portion of the jacket for purposes of permitting easy access of nutrients from the patient into the cell or of entry of the patient's proteins into the cell to be acted upon by the cell to provide a metabolic function. In that regard, a non-spherical shape, such as a cylinder, is preferred.

Four important factors that influence the number of cells or amount of tissue to be placed within the core of the device (i.e., loading density) of the instant invention are: (1) device size and geometry; (2) mitotic activity within the device; (3) viscosity requirements for core preparation and or loading; and (4) pre-implantation assay and qualification requirements.

With respect to the first of these factors, (device size and geometry), the diffusion of critical nutrients and metabolic requirements into the cells as well as diffusion of metabolites away from the cell are critical to the continued viability of the cells. In the case of RPE cells such as ARPE-19 cells, the neighboring cells are able to phagocytize the dying cells and use the debris as an energy source.

Among the metabolic requirements met by diffusion of substances into the device is the requirement for oxygen. The oxygen requirements of the specific cells must be determined for the cell of choice. See Methods and references for determination of oxygen metabolism are given in Wilson D. F. et al., J. Biol. Chem., 263, pp. 2712-2718, (1988).

With respect to the second factor (cell division), if the cells selected are expected to be actively dividing while in the device, then they will continue to divide until they fill the available space, or until phenomena such as contact inhibition limit further division. For replicating cells, the geometry and size of the device will be chosen so that complete filling of the device core will not lead to deprivation of critical nutrients due to diffusional limitations.

With respect to the third factor (viscosity of core materials) cells in densities occupying up to 70% of the device volume can be viable, but cell solutions in this concentration range would have considerable viscosity. Introduction of cells in a very viscous solution into the device could be prohibitively difficult. In general, for both two step and coextrusion strategies, cell loading densities of higher than 30% will seldom be useful, and in general optimal loading densities will be 20% and below. For example, for fragments of tissues, it is important, in order to preserve the viability of interior cells, to observe the same general guidelines as above and tissue fragments should not exceed 250 microns in diameter with the interior cells having less than 15, preferably less than 10 cells between them and the nearest diffusional surface.

Finally, with respect to the fourth factor (preimplantation and assay requirements), in many cases, a certain amount of time will be required between device preparation and implantation. For instance, it may be important to qualify the device in terms of its biological activity. Thus, in the case of mitotically active cells, preferred loading density will also consider the number of cells which must be present in order to perform the qualification assay.

In most cases, prior to implantation in vivo, it will be important to use in vitro assays to establish the efficacy of the BAM (e.g., PEDF) within the device. Devices can be constructed and analyzed using model systems in order to allow the determination of the efficacy of the vehicle on a per cell or unit volume basis.

Following these guidelines for device loading and for determination of device efficacy, the actual device size for implantation will then be determined by the amount of biological activity required for the particular application. The number of devices and device size should be sufficient to produce a therapeutic effect upon implantation and is determined by the amount of biological activity required for the particular application. In the case of secretory cells releasing therapeutic substances, standard dosage considerations and criteria known to the art will be used to determine the amount of secretory substance required. Factors to be considered include the size and weight of the recipient; the productivity or functional level of the cells; and, where appropriate, the normal productivity or metabolic activity of the organ or tissue whose function is being replaced or augmented. It is also important to consider that a fraction of the cells may not survive the immunoisolation and implantation procedures. Moreover, whether the recipient has a preexisting condition which can interfere with the efficacy of the implant must also be considered.

Devices of the instant invention can easily be manufactured which contain many thousands of cells. For example, current clinical devices contain between 200,000 and 400,000 cells, whereas micronized devices would contain between 10,000 and 100,000 cells.

Encapsulated cell therapy is based on the concept of isolating cells from the recipient host's immune system by surrounding the cells with a semipermeable biocompatible material before implantation within the host. For example, the invention includes a device in which genetically engineered ARPE-19 cells are encapsulated in an immunoisolatory capsule, which, upon implantation into a recipient host, minimizes the deleterious effects of the host's immune system on the ARPE-19 cells in the core of the device. ARPE-19 cells are immunoisolated from the host by enclosing them within implantable polymeric capsules formed by a microporous membrane. This approach prevents the cell-to-cell contact between the host and implanted tissues, thereby eliminating antigen recognition through direct presentation.

PEDF can be delivered intraocularly (e.g., in the anterior chamber and the vitreous cavity) or periocularly (e.g., within or beneath Tenon's capsule), or both. The devices of the invention may also be used to provide controlled and sustained release of PEDF to treat various ophthalmic disorders, ophthalmic diseases and/or diseases which have ocular effects.

The present invention provides methods for the treatment or prevention of ophthalmic diseases and disorders by implanting the encapsulated PEDF-secreting cells described herein into the eye. According to the present methods, the encapsulated cells are implanted intraocularly or periocularly. In one embodiment, the cells are implanted intraoculalry into the vitreous. In another embodiment, the cells are implanted periocularly, into the sub-Tenon's region of the eye.

Ophthalmic diseases and disorders that can be treated or prevented using the encapsulated PEDF-secreting cells of the invention include those characterized by neovascularization and/or accumulation of fluid within the layers of the eye and within the vitreal cavity. Ocular neovascularization is one of the most common causes of blindness and underlies the pathology of a number of eye diseases. Retinal ischemia-associated ocular neovascularization is a major cause of blindness in diabetes and other diseases. For example, in diabetes, new capillaries formed in the retina invade the vitreous humor, causing bleeding and blindness. Thus, diabetic retinopathy is characterized by aberrant angiogenesis.

In one embodiment, the PEDF-secreting encapsulated cells of the invention are used for the treatment of diabetic retinopathy. In accordance with this embodiment, the cells are implanted intraocularly, preferably in the vitreous, or periocularly, preferably in the sub-Tenon's region. In a preferred embodiment, the cells are implanted in the vitreous for the treatment of diabetic retinopathy. In one embodiment, the PEDF-secreting encapsulated cells form part of a treatment regimen that includes the administration of one or more additional therapeutic agents. Preferably, the one or more additional therapeutic agents is a neurotrophic factor.

Other ocular-related diseases characterized by neovascularization that can be treated with the PEDF-secreting encapsulated cells of the invention include, without limitation, corneal neovascularization, choroidal neovascularization, neovascular glaucoma, cyclitis, Hippel-Lindau Disease, retinopathy of prematurity, pterygium, histoplasmosis, iris neovascularization, macular edema, and glaucoma-associated neovascularization. Neovascularization is also associated with central retinal vein occlusion and sometimes age-related macular degeneration. Corneal neovascularization is a major problem because it interferes with vision and predisposes patients to corneal graft failure. A majority of severe visual loss is associated with disorders that results in ocular neovascularization.

Vascular leakage can cause retinal detachment, degeneration of sensory cells of the eye, increased intraocular pressure, and inflammation, all of which adversely affect vision and the general health of the eye. Exemplary diseases and disorders characterized by accumulation of fluid or vascular leakage that can be treated with the PEDF-secreting encapsulated cells of the invention include, without limitation, nonproliferative diabetic retinopathy, proliferative retinopathies, retinopathy of prematurity, retinal vascular diseases, vascular anomalies, choroidal disorders, choroidal neovascularization, neovascular glaucoma, glaucoma, macular edema (e.g., diabetic macular edema), retinal edema (e.g., diabetic retinal edema), central serous chorioretinopathy, and retinal detachment caused by accumulation of vascular fluid within the layers of the eye.

Those skilled in the art will recognize that other ophthalmic disorders that may be treated by various embodiments of the present invention include, but are not limited to, diabetic retinopathies, diabetic macular edema, proliferative retinopathies, retinal vascular diseases, vascular anomalies, age-related macular degeneration and other acquired disorders, endophthalmitis, infectious diseases, inflammatory but non-infectious diseases, AIDS-related disorders, ocular ischemia syndrome, pregnancy-related disorders, peripheral retinal degenerations, retinal degenerations, toxic retinopathies, retinal tumors, choroidal tumors, choroidal disorders, vitreous disorders, retinal detachment and proliferative vitreoretinopathy, non-penetrating trauma, penetrating trauma, post-cataract complications, and inflammatory optic neuropathies. In addition, those skilled in the art will recognize that retinal degenerative disorders, including, but not limited to, retinitis pigmentosa, glaucoma, age-related macular degeneration, diabetic macular edema, and diabetic retinopathy can also be treated using the capsules of the invention.

Age-related macular degeneration (AMD) is one of the most common causes of vision loss among adults in the U.S. The form of the disease most often progressing to blindness is characterized by detachment of the retinal pigment epithelium and choroidal neovascularization (CNV). The damage caused by the leakage and fibrovascular scarring leads to profound loss of central vision and severe loss of visual acuity. Age-related macular degeneration includes, without limitation, dry age-related macular degeneration, exudative age-related macular degeneration, and myopic degeneration.

In some preferred embodiments, the disorder to be treated is the wet form of age-related macular degeneration or diabetic retinopathy. The present invention may also be useful for the treatment of ocular neovascularization, a condition associated with many ocular diseases and disorders. For example, retinal ischemia-associated ocular neovascularization is a major cause of blindness in diabetes and many other diseases.

The devices of the present invention may also be used to treat ocular symptoms resulting from diseases or conditions that have both ocular and non-ocular symptoms. Some examples include cytomegalovirus retinitis in AIDS as well as other conditions and vitreous disorders; hypertensive changes in the retina as a result of pregnancy; and ocular effects of various infectious diseases such as tuberculosis, syphilis, lyme disease, parasitic disease, toxocara canis, ophthalmonyiasis, cyst cercosis and fungal infections.

The invention also relates to methods and the delivery of PEDF in order to treat cell proliferative disorders, such as, hematologic disorders, atherosclerosis, inflammation, increased vascular permeability, and malignancy.

In addition, those skilled in the art will also recognize that the invention also relates to methods and the delivery of PEDF as a neuroprotective factor. In particular, because PEDF facilitates cell movement into a quiescent phase in the cell cycle, aids in differentiation, and protects neurons from damage (see Tombran-Tink, Frontiers in Bioscience 10:2131-2149 (2005)), the capsules and methods described herein can also be used in the treatment of diseases and disorders characterized by neural or retinal damage and/or degradation.

The use of the devices and techniques described herein provide several advantages over other delivery routes. Specifically, PEDF can be delivered to the eye directly, which reduces or minimizes unwanted peripheral side effects and very small doses of the biologically active molecule (i.e., nanogram or low microgram quantities rather than milligrams) can be delivered compared with topical applications, thereby also potentially lessening side effects. Moreover, since viable cells continuously produce newly synthesized biologically active molecules, these techniques should be superior to injection delivery of PEDF, where the dose fluctuates greatly between injections and the biologically active molecule is continuously degraded but not continuously replenished.

Living cells and cell lines genetically engineered to secrete PEDF can be encapsulated in the device of the invention and surgically inserted (under retrobulbar anesthesia) into any appropriate anatomical structure of the eye. For example, the devices can be surgically inserted into the vitreous of the eye, where they are preferably tethered to the sclera to aid in removal. Devices can remain in the vitreous as long as necessary to achieve the desired prophylaxis or therapy. For example, the desired therapy may include promotion of neuron or photoreceptor survival or repair, or inhibition and/or reversal of retinal or choroidal neovascularization, as well as inhibition of uveal, retinal and optic nerve inflammation. With vitreal placement, PEDF may be delivered to the retina or the retinal pigment epithelium (RPE).

In other embodiments, cell-loaded devices are implanted periocularly, within or beneath the space known as Tenon's capsule, which is less invasive than implantation into the vitreous. Therefore, complications such as vitreal hemorrhage and/or retinal detachment are potentially eliminated. This route of administration also permits delivery of PEDF to the RPE or the retina. Periocular implantation is especially preferred for treating choroidal neovascularization and inflammation of the optic nerve and uveal tract. In general, delivery from periocular implantation sites will permit circulation of PEDF to the choroidal vasculature, retinal vasculature, and the optic nerve.

Delivery of anti-angiogenic factors, such as PEDF of the invention, directly to the choroidal vasculature (periocularly) or to the vitreous (intraocularly) using the devices and methods described herein may reduce or alleviate the problems associated with prior art treatment methods and devices and may permit the treatment of poorly defined or occult choroidal neovascularization as well as provide a way of reducing or preventing recurrent choroidal neovascularization via adjunctive or maintenance therapy.

The encapsulated cell devices are implanted according to known techniques, preferably into the aqueous and vitreous humors of the eye. (See WO97/34586). Implantation of the biocompatible devices of the invention is performed under sterile conditions. The device can be implanted using a syringe or any other method known to those skilled in the art. Generally, the device is implanted at a site in the recipient's body which will allow appropriate delivery of the secreted product or function to the recipient and of nutrients to the implanted cells or tissue, and will also allow access to the device for retrieval and/or replacement. A number of different implantation sites are contemplated. These include, e.g., the aqueous humor, the vitreous humor, the sub-Tenon's capsule, the periocular space, and the anterior chamber. Preferably, for implant sites that are not immunologically privileged, such as periocular sites, and other areas outside the anterior chamber (aqueous) and the posterior chamber (vitreous), the capsules are immunoisolatory.

It is preferable to verify that the cells immobilized within the device function properly both before and after implantation. Any assays or diagnostic tests well known in the art can be used for these purposes. For example, an ELISA (enzyme-linked immunosorbent assay), chromatographic or enzymatic assay, or bioassay specific for the secreted product can be used. If desired, secretory function of an implant can be monitored over time by collecting appropriate samples (e.g., serum) from the recipient and assaying them.

The use of many of the prior art devices and surgical techniques resulted in a large number of retinal detachments. The occurrence of this complication is lessened because the devices and methods of this invention are less invasive compared to several other therapies.

Modified, truncated and/or mutein forms of PEDF can also be used in accordance with this invention. Further, the use of active fragments of PEDF (i.e., those fragments having biological activity sufficient to achieve a therapeutic effect) is also contemplated. Also contemplated is the use of PEDF modified by attachment of one or more polyethylene glycol (PEG) or other repeating polymeric moieties as well as combinations of these proteins and polycistronic versions thereof.

In accordance with certain embodiments of the methods of the invention, the encapsulated cells are surgically implanted into the vitreous of the eye. Preferably, the entire body of the capsule containing the cells is implanted in the vitreous, however a portion of the capsule may protrude, e.g., into or through the sclera. Preferably the device is tethered to the sclera or other suitable ocular structure. In a specific embodiment, the tether comprises a suture eyelet or disk.

In other embodiments, the encapsulated cells are implanted periocularly, within or beneath the space known as Tenon's capsule. This embodiment is less invasive than implantation into the vitreous and thus is generally preferred. This route of administration also permits delivery of PEDF to the RPE or the retina. This embodiment is especially preferred for treating choroidal neovascularization and inflammation of the optic nerve and uveal tract. In general, delivery from this implantation site will permit circulation of PEDF to the choroidal vasculature, the retinal vasculature, and the optic nerve.

Treatment of many conditions according to the methods described herein will require only one or at most less than 50 implanted devices per eye to supply an appropriate therapeutic dose. Therapeutic dosages may be between about 0.1 pg and 1000 ng per eye per patient per day (e.g., between 0.1 pg and 500 ng per eye per patient per day; between 0.1 pg and 250 ng, between 0.1 pg and 100 ng, between 0.1 pg and 50 ng, between 0.1 pg and 25 ng, between 0.1 pg and 10 ng, or between 0.1 pg and 5 ng per eye per patient per day). Each of the devices of the present invention is capable of storing between about 10² and 10⁸ cells, most preferably 5×10² to 5×10⁵ cells (e.g., ARPE-19 cells) that have been genetically engineered to secreted PEDF.

In one embodiment, the encapsulated PEDF-secreting cells of the invention are used for the treatment of age-related macular degeneration to deliver PEDF intraocularly, preferably to the vitreous, or periocularly, preferably to the sub-Tenon's region. In a further embodiment, the PEDF-secreting encapsulated cells form part of a treatment regimen that includes the administration of one or more additional therapeutic agents. Preferably, the one or more additional therapeutic agents is a neurotrophic factor.

In certain embodiments, the PEDF-secreting encapsulated cells of the invention are administered as part of a therapeutic regimen that includes the administration of one or more additional therapeutic agents. In certain embodiments, the one or more additional therapeutic agents is an anti-inflammatory factor or a neurotrophic factor. As used herein, a neurotophic factor is one that retards cell degeneration, promotes cell sparing, or promotes new cell growth.

Preferably, the one or more additional therapeutic agents is administered either intraocularly or periocularly, preferably intraocularly, and most preferably intravitreally. In certain embodiments, the one or more additional therapeutic agents is administered at the same time, or at substantially the same time as the PEDF-secreting encapsulated cells are implanted.

In one embodiment, the one or more additional therapeutic agents is administered at substantially the same time as PEDF. In a specific embodiment, the cells are transfected with separate constructs encoding PEDF and the therapeutic agent, or the cells are transfected with a single construct encoding both PEDF and the therapeutic agent. Techniques for multiple gene expression from a single transcript are known in the art and are preferred over expression from multiple transcription units. See e.g., Macejak, Nature (1991) 353:90-94; Mountford and Smith, (1995) Trends Genet., 11:179-184; Dirks et al., Gene, (1993) 128:24-49; Martinez-Salas et al., J. Virology, (1993) 67:3748-3755; Mountford et al., Proc. Natl. Acad. Sci. U.S.A., (1994) 91:4303-4307; and PCT International Application Publication No. WO 94/24870.

In another embodiment, either two or more separately engineered cell lines are co-encapsulated. In another embodiment, more than one device is implanted either at the same or in different sites in the eye concurrently, to deliver PEDF and one or more additional therapeutic agents. In a specific embodiment, a neurotrophic factor is delivered to the vitreous of the eye to supply the neural retina (ganglion cells to the RPE) and PEDF is delivered to the sub-Tenon's space to supply the choroidal vasculature. In certain embodiments, 1, 2, 3, 4, or 5 devices comprising encapsulated cells secreting PEDF and/or one or more additional therapeutic agents is implanted per eye. Preferably, 1 to 3 devices is implanted per eye.

In one embodiment, the one or more additional therapeutic agents is an anti-inflammatory factor selected from an antiflammin (see e.g., U.S. Pat. No. 5,266,562), beta-interferon (IFN-β), alpha-interferon (IFN-α), TGF-beta, interleukin-10 (IL-10), a glucocorticoid, or a mineralocorticoid.

In one embodiment, the one or more additional therapeutic agents is a neurotrophic factor selected from neurotrophin 4/5 (NT-4/5), cardiotrophin-1 (CT-1), ciliary neurotrophic factor (CNTF), glial cell line derived neurotrophic factor (GDNF), nerve growth factor (NGF), insulin-like growth factor-1 (IGF-1), neurotrophin 3 (NT-3), brain-derived neurotrophic factor (BDNF), PDGF, neurturin, acidic fibroblast growth factor (aFGF), basic fibroblast growth factor (FGF), EGF, neuregulins, heregulins, TGF-alpha, bone morphogenic proteins (BMP-1, BMP-2, BMP-7, etc.), the hedgehog family (sonic hedgehog, indian hedgehog, and desert hedgehog, etc.), the family of transforming growth factors (including, e.g., TGFβ-1, TGFβ-2, and TGFβ-3), interleukin 1-B (IL1-β), and such cytokines as interleukin-6 (IL-6), IL-10, CDF/LIF, and beta-interferon (IFN-β). Preferably, the neurotrophic factor is selected from GDNF, BDNF, NT-4/5, neurturin, CNTF, and CT-1.

The dose of PEDF to be administered intraocularly, preferably in the vitreous, is in the range of 50 picograms to 500 nanograms, preferably from 100 picograms to 100 nanograms, and most preferably 1 nanogram to 50 nanograms per eye per patient per day. For periocular delivery, preferably in the sub-Tenon's space or region, slightly higher dosage ranges are contemplated of up to 1 microgram per patient per day. For example, current clinical devices result in vitreal levels of 1-500 ng (pre-implantation or in vitro). Explanted devices (post-in vivo) have been shown to release 10-500 ng/device/day.

Dosage can be varied, for example, by changing (1) the number of cells per device, (2) the number of devices per eye, or (3) the level of PEDF production per cell. Cellular production can be varied by changing, for example, the copy number of the gene for PEDF in the cells, or the efficiency of the promoter driving expression of PEDF. Preferably, about 10³ to 10⁸ cells are encapsulated per device, more preferably from about 5×10⁴ to 5×10⁶ cells per device.

The invention will be further described in the following examples, which do not limit the scope of the invention described in the claims.

### EXAMPLES

### Example 1

### Sub-Cloning

cDNA encoding human PEDF (GenBank Accession No. NM_002615) was subcloned into Neurotech mammalian expression vector pKAN2, a schematic of which is shown in FIG. 1. The pKAN2 backbone is based on the pNUT-IgSP-hCNTF expression plasmid used to create the ARPE-19-hCNTF cell lines.

The nucleotide sequence of pKAN2 is shown below in SEQ ID NO: 3:
cttggtttttaaaaccagcctggagtagagcagatgggttaaggtgagtgacccctcagccctggacattcttagatgagccccctcaggagt
agagaataatgttgagatgagttctgttggctaaaataatcaaggctagtctttataaaactgtctcctcttctcctagcttcgatccagagagag
acctgggcggagctggtcgctgctcaggaactccaggaaaggagaagctgaggttaccacgctgcgaatgggtttacggagatagctgg
ctttccggggtgagttctcgtaaactccagagcagcgataggccgtaatatcggggaaagcactatagggacatgatgttccacacgtcaca
tgggtcgtcctatccgagccagtcgtgccaaaggggcggtcccgctgtgcacactggcgctccagggagctctgcactccgcccgaaaa
gtgcgctcggctctgccaaggacgcggggcgcgtgactatgcgtgggctggagcaaccgcctgctgggtgcaaaccctttgcgcccgga
ctcgtccaacgactataaagagggcaggctgtcctctaagcgtcacccctagagtcgagctgtgacggtccttacactcgagaccggtgcg
gccgcatttaaatactagtccgggtggcatccctgtgacccctccccagtgcctctcctggccctggaagttgccactccagtgcccaccag
ccttgtcctaataaaattaagttgcatcattttgtctgactaggtgtccttctataatattatggggtggaggggggtggtatggagcaaggggc
aagttgggaagacaacctgtagggcctgcggggtctattgggaaccaagctggagtgcagtggcacaatcttggctcactgcaatctccgc
ctcctgggttcaagcgattctcctgcctcagcctcccgacggccgtaattcgtaatcatgtcatagctgtttcctgtgtgaaattgttatccgctc
acaattccacacaacatacgagccggaagcataaagtgtaaagcctggggtgcctaatgagtgagctaactcacattaattgcgttgcgctc
actgcccgctttccagtcgggaaacctgtcgtgccagctgcattaatgaatcggccaacgcgcggggagaggcggtttgcgtattgggcgc
tatccgcttcctcgctcactgactcgctgcgctcggtcgttcggctgcggcgagcggtatcagctcactcaaaggcggtaatacggttatcc
acagaatcaggggataacgcaggaaagaacatgtgagcaaaaggccagcaaaaggccaggaaccgtaaaaaggccgcgttgctggcg
tttttccataggctccgcccccctgacgagcatcacaaaaatcgacgctcaagtcagaggtggcgaaacccgacaggactataaagatacc
aggcgtttccccctggaagctccctcgtgcgctctcctgttccgaccctgccgcttaccggatacctgtccgcctttctcccttcgggaagcgt
ggcgctttctcatagctcacgctgtaggtatctcagttcggtgtaggtcgttcgctccaagctgggctgtgtgcacgaaccccccgttcagcc
cgaccgctgcgccttatccggtaactatcgtatgagtccaacccggtaagacacgacttatcgccactggcagcagccactggtaacagg
attagccagagcgaggtatgtaggcggtgctacagagttcttgaagtggtggcctaactacggctacactagaagaacagtatttggtatctg
cgctctgctgaagccagttaccttcggaaaaagagttggtagctcttgatccggcaaacaaaccaccgctggtagcggtggtttttttgtttgc
aagcagcagattacgcgcagaaaaaaaggatctcaagaagatcctttgatcttttctacggggtctgacgctcagtggaacgaaaactcacg
ttaagggattttggtcatgagattatcaaaaaggatcttcacctaaatccttttaaattaaaaatgaagttttaaatcaatctaaagtatatatgagta
aacttggtctgacagtctaagaaaccattattatcatgacattaacctataaaaataggcgtatcacgaggccctttcgtctcgcgcgtttcggt
gatgacggtgaaaacctctgacacatgcagctcccggagacggtcacagcttgtctgtaagcggatgccgggagcagacaagcccgtca
gggcgcgtcagcgggtgttggcgggtgtcggggctggcttaactatgcggcatcagagcagattgtactgagagtgcaccgatccccccg
gtacccgatccagacatgataagatacattgatgagtttggacaaaccacaactagaatgcagtgaaaaaaatgctttatttgtgaaatttgtga
tgctattgctttatttgtaaccattataagctgcaataaacaagttaacaacaacaattgcattcattttatgtttcaggttcagggggaggtgtgg
gaggttttttaaagcaagtaaaacctctacaaatgtggtatggctgattatgatcatgaacagactgtgaggactgaggggcctgaaatgagc
cttgggactgtgaatctaaaatacacaaacaattagaatcagtagtttaacacattatacacttaaaaattttatatttaccttagagetttaaatctc
tgtaggtagtttgtccaattatgtcacaccacagaagtaaggttccttcacaaagatcccaagtcgaaccccagagtcccgctcagaagaact
cgtcaagaaggcgatagaaggcgatgcgctgcgaatcgggagcggcgataccgtaaagcacgaggaagcggtcagcccattcgccgc
caagctcttcagcaatatcacgggtagccaacgctatgtcctgatagcggtccgccacacccagccggccacagtcgatgaatccagaaa
agcggccattttccaccatgatattcggcaagcaggcatcgccatgggtcacgacgagatcctcgccgtcgggcatgcgcgccttgagcct
ggcgaacagttcggctggcgcgagcccctgatgctcttcgtccagatcatcctgatcgacaagaccggcttccatccgagtacgtgctcgct
cgatgcgatgtttcgcttggtggtcgaatgggcaggtagccggatcaagcgtatgcagccgccgcattgcatcagccatgatggatactttct
cggcaggagcaaggtgagatgacaggagatcctgccccggcacttcgcccaatagcagccagtcccttcccgcttcagtgacaacgtcg
agcacagctgcgcaaggaacgcccgtcgtggccagccacgatagccgcgctgcctcgtcctgcagttcattcagggcaccggacaggtc
ggtcttgacaaaaagaaccgggcgcccctgcgctgacagccggaacacggcggcatcagagcagccgattgtctgttgtgcccagtcata
gccgaatagcctctccacccaagcggccggagaacctgcgtgcaatccatcttgttcaatcatgcgaaacgatcctcatcctgtctcttgatc
agatcccaagctggggatctgcaggaatcgatatcaagcttatcgataagctttttgcaaaagcctaggcctccaaaaaagcctcctcactac
ttctggaatagctcagaggccgaggcggcctcggcctctgcataaataaaaaaaattagtcagccatggggcggagaatgggcggaactg
ggcggagttaggggcgggatgggcggagttaggggcgggactatggttgctgactaattgagatgcatgctttgcatacttctgcctgctgg
ggagcctggggactttccacacctggttgctgactaattgagatgcatgctttgcatacttctgcctgctggggagcctggggactttccacac
cctaactgacacacattccacagctggttctttccgcctcagaaggtacactcttcctttttcaatattattgaagcatttatcagggttattgtctca
tgagcggatacatatttgaatgtatttagaaaaataaacaaataggggttccgcgcacatttccccgaaaagtgccacctgacggcggcc

Transformed recombinant clones were selected with kanamycin, and purified miniprep plasmid DNA was analyzed by restriction digestion and agarose gel electrophoresis analysis. Putative plasmid clones containing an appropriate insert were verified by automated dideoxy sequencing followed by alignment analysis using Vector NTI v7.0 sequence analysis software (Invitrogen Corp, Carlsbad, Calif.).

### Example 2

### Cell Line and Device Construction

Verified plasmid clones were used to transfect NTC-200 cells to obtain stable polyclonal cell lines. Briefly, 200-300K cells, plated 18 hours previously, were transfected with 3.0 ug of plasmid DNA using 6.0 ul of Fugene 6 transfection reagent (Roche Applied Science, Indianapolis Ind.) according to the manufacturer's recommendations. Transfections were performed in 3.0 ml of DMEM/F12 with 10% FBS, Endothelial SFM or Optimem media (Invitrogen Corp, Carlsbad, Calif.). Twenty four to 48 hours later cells were either fed with fresh media containing 1.0 ug/ul of G418 or passaged to a T-25 tissue culture flask containing G418. Cell lines were passaged under selection for 14-21 days until normal growth resumed, after which time drug was removed and cells were allowed to recover (˜1 week) prior to characterization.

Expression stability of the recombinant protein from these cell lines was measure over the course of several weeks using the Human PEDF Sandwich ELISA Antigen Detection Kit (BioProducts MD, Middletown, Md.). Briefly, 50K cells, previously plated into 12 well tissue culture plates in DMEM/F12 with 10% FBS, were washed twice in HBSS (Invitrogen Corp, Carlsbad, Calif.) then pulsed for 2 hours with 1.0 ml of Endothelial SFM (Invitrogen Corp, Carlsbad, Calif.). Pulse media was stored at −20 C and assayed within one week of collection as per the manufacturer's protocol.

Thus, stable cell lines secreting PEDF were successfully created.

Candidate engineered lines are screened for expression levels of the protein of interest prior to encapsulation. In general, 50 k cells are pulsed for 2 hours at 37 C and the resulting conditioned media is assayed, usually by ELISA. Protein expression is reported as ng/million cells/24 hours. In the case of PEDF high-producing lines express 1000-10000 ng/million cells/day.

These cell lines were packaged into encapsulated cell therapy devices in accordance with this invention. The secretion of PEDF from the devices was monitored. Devices secreting therapeutic levels of PEDF were used for further studies.

### Example 3

### Protein Characterization

Expression of PEDF from stably transfected cell lines was quantified by analyzing conditioned media from cell monolayers using a commercially available ELISA kit (BioProducts Maryland, Middletown, Md.). Conditioned media from stably transfected cells was analyzed by a colorimetric Western blot assay to determine protein integrity.

### Example 4

### A Safety and Feasibility Study of ECT Devices Secreting PEDF

(NT-502) Vitreous Cavity Implantation in Patients with Clinically Significant Macular Edema (CSME) Secondary to Diabetes Mellitus (Type 1 or 2)

Diabetic Macular Edema (DME) is a leading cause of blindness. It is a complication of diabetic retinopathy that leads to progressive vision loss. In DME, VEGF upregulation leads to new vessel growth, vessel dysfunction, and fluid leakage in the macula.

Primary Objectives

The primary objectives of this study included the evaluation of safety and tolerability of NT-502 vitreous cavity implantation and the evaluation of the efficacy of NT-502 vitreous cavity implantation, as measured by the percentage of subjects gaining ≧15 letters in best corrected visual acuity (BCVA) from baseline.

Additional Objectives

Additional objectives included the evaluation of the safety and tolerability of NT-502 vitreous cavity implantation through the collection of adverse events and serious adverse events and ocular assessments and the evaluation of the efficacy of the efficacy of NT-502 vitreous cavity implantation with respect to BCVA outcomes, anatomic outcomes, and patient-reported visual functioning outcomes over a 12-month period in subjects with CSME.

Study Design

This study was a Phase I, single dose, open-label, prospective non-randomized, single center, pilot study to evaluate the safety of NT-502 vitreous cavity implantation in patients with CSME secondary to diabetes mellitus (Type 1 or 2). Nine subjects were included in one investigational center in Mexico. This study consisted of a screening period of up to 7 weeks (Days—7 weeks to—day 1) and a treatment day (implant day 0). The duration of the study was 12 months, excluding the screening period.

Subjects who provided consent entered the screening period to determine eligibility. As part of the screening process, the investigators evaluated macular optical coherence tomography (OCT) images to determine subjects' eligibility. Eligible subjects were treated with NT-502 vitreous cavity implantation.

Subjects met BCVA and retinal thickness eligibility requirements during both the screening period and on Day 0. Determination of a subject's eligibility on Day 0 was made by the evaluating physician. Only one eye was chosen as the study eye. If both eyes were eligible, the eye with the worse VA as assessed at screening was selected for study treatment unless, based on medical reasons, the investigator deemed the other eye to be more appropriate for treatment and study. Only the study eye was treated. The non-study eye may have received laser photocoagulation for CSME consistent with the standard of care.

Subjects had 10 scheduled visits during the 12-month study for the evaluation of safety and efficacy. Subjects had surgical implantation of NT-502 in the vitreous cavity on Day 0 and underwent safety and ocular assessments by the evaluating physician.

Each subject's study eye were evaluated for the need for macular laser treatment (standard care) beginning at the Month 3 visit and as needed thereafter based on the protocol-defined criteria. Subjects with bilateral DME may have received standard of care laser therapy in the fellow (non-study) eye no sooner than 1 day preceding or following macular laser and/or study treatment (implant) in the study eye.

### Outcomes

Primary Outcome

The primary outcome was the safety of the implantation of the NT-502 device. The safety of the NT-502 device was assessed by the following outcomes, occurrence of these outcomes did not necessarily require explanation.
1. local adverse events
2. cataract progression
3. severe TOP changes
4. infectious endophthalmitis
5. severe ocular inflammation
6. retinal detachment
7. severe VA loss (>3 lines ETDRS)
8. progression of diabetic retinopathy
9. vitreous hemorrhage
10. implant related (extrusion, erosion, etc)
11. systemic adverse events
12. abnormal findings from serum chemistry, hematology, and urinalysis/urine chemistry (abnormal implying out of range findings, or of clinical chemistry toxicities of Grade II or higher)
13. symptoms of immune disorders or allergy

Secondary Outcome

The secondary outcome measures related to potential product performance were:
1. The proportion of subjects who gain at least 15 letters in BCVA compared with baseline at 6 and 12 months post treatment.
2. Mean change from baseline in BCVA score over time at 6 and 12 months
3. Mean change from baseline in central foveal thickness (CFT) over time at 6 and 12 months, as assessed on OCT
4. Proportion of subjects with resolution of leakage at 6 and 12 months, using fluorescein angiography (FA)
5. Mean number of macular laser treatments up to 12 months
6. Mean change from baseline in the National Eye Institute Visual Functioning Questionnaire-25 (NEI VFQ-25) near activities subscale score over time at 6 and 12 months
7. Proportion of subjects with a three-step change from baseline in the Early Treatment Diabetic Retinopathy Study (ETDRS) scale at 6 and 12 months
8. Mean change from baseline in contrast sensitivity at 6 and 12 months

### Subject Selection

Subjects with CSME secondary to diabetes mellitus (Type 1 or 2) were enrolled in the study. Written informed consent was obtained before initiation of any study procedures. Screening evaluations were performed at any time within the 7 weeks preceding Day 0 (the day of implant).

### Inclusion Criteria

Subjects must have met the following criteria to be eligible for study entry:
1. Willingness to provide written informed consent.
2. Age ≧18 years
3. Diabetes mellitus (Type 1 or 2)
4. Any of the following were considered as sufficient evidence that diabetes was present:
   a. Current regular use of insulin for the treatment of diabetes
   b. Current regular use of oral antihyperglycemic agents for the treatment of diabetes
   c. Documented diabetes
5. Retinal thickening secondary to diabetes mellitus (DME) involving the center of the fovea with central macular thickness >275 μm in the center subfield, as assessed on OCT at the screening visit only and by the evaluating physician on Day 0
6. BCVA score in the study eye of 20/40 to 20/320 approximate Snellen equivalent
7. Decrease in vision determined to be primarily the result of DME and not to other causes
8. For sexually active women of childbearing potential, use of an appropriate form of contraception (or abstinence) for the duration of the study
9. Ability (in the opinion of the investigator) and willingness to return for all scheduled visits and assessments

### Exclusion Criteria

Subjects who met any of the following criteria were excluded from study entry:

### Ocular Conditions

Prior and Concomitant Treatments
1. History of vitreoretinal surgery in the study eye
2. Panretinal photocoagulation (PRP) or macular laser photocoagulation in the study eye within 6 months of screening
3. Previous use of any intraocular drug in the study or fellow eye (pegaptanib sodium, anecortave acetate, bevacizumab, ranibizumab, etc.)

Diabetic Retinopathy Characteristics
4. PDR in the study eye, with the exception of inactive, regressed PDR
5. Iris neovascularization, vitreous hemorrhage, traction retinal detachment, or preretinal fibrosis involving the macula in the study eye

Concurrent Ocular Conditions
6. Vitreomacular traction or epiretinal membrane in the study eye evident biomicroscopically or on OCT that is considered by the investigator to significantly affect central vision
7. Ocular inflammation in the study eye
8. History of idiopathic or autoimmune uveitis in either eye
9. Structural damage to the center of the macula in the study eye that is likely to preclude improvement in VA following the resolution of macular edema, including atrophy of the RPE, subretinal fibrosis, or organized hard-exudate plaque.
10. Ocular disorders in the study eye that may confound interpretation of study results, including retinal vascular occlusion, retinal detachment, macular hole, or CNV of any cause (e.g., AMD, ocular histoplasmosis, or pathologic myopia, secondary to laser)
11. Concurrent disease in the study eye that would compromise VA or require medical or surgical intervention during the study period
12. Cataract surgery in the study eye within 3 months, yttrium-aluminum-garnet (YAG) laser capsulotomy within the past 2 months, or any other intraocular surgery within the 90 days preceding Day 0.
13. Uncontrolled glaucoma (defined as TOP >□30 mmHg despite treatment with antiglaucoma medication) or previous filtration surgery in the study eye
14. Spherical equivalent of the refractive error in the study eye of more than 6 diopters myopia (for subjects who have had refractive or cataract surgery in the study eye, preoperative spherical equivalent refractive error of more than 6 diopters myopia)
15. Axial length >26 mm by A-scan ultrasound
16. Evidence at examination of infectious blepharitis, keratitis, scleritis or conjunctivitis in either eye, or current treatment for serious systemic infection

### Systemic Conditions or Treatments

17. Uncontrolled blood pressure (defined as systolic >□180 mmHg and diastolic 110 mmHg while subject is sitting)
18. History of cerebral vascular accident or myocardial infarction
19. Uncontrolled diabetes mellitus, evidenced by a HbAlc value of 12%
20. Renal failure requiring dialysis or renal transplant
21. History of participation in an investigational trial that involved treatment with any drug (excluding vitamins and minerals) or device
22. History of other disease, metabolic dysfunction, physical examination finding, or clinical laboratory finding giving reasonable suspicion of a disease or condition that contraindicates the use an investigational drug, might affect interpretation of the results of the study, or renders the subject at high risk from treatment complications
23. Pregnancy or lactation
24. History of allergy to fluorescein
25. Inability to obtain fundus photographs or fluorescein angiograms of sufficient quality to be analyzed and graded by the central reading center
26. Inability to comply with study or follow-up procedures

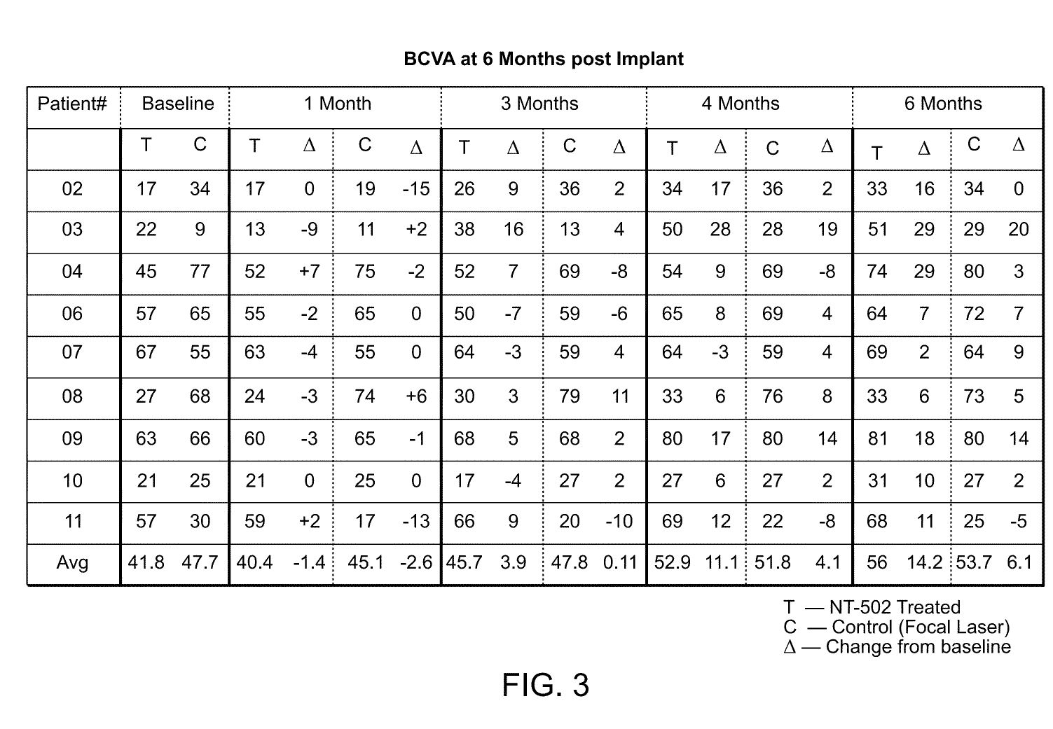

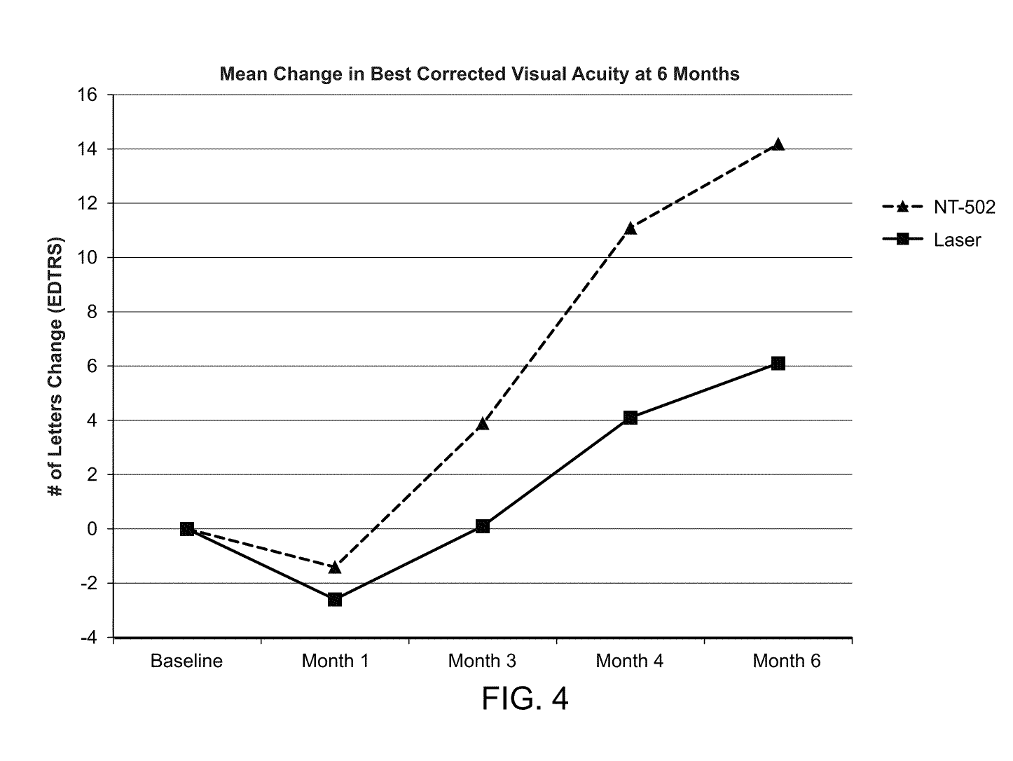

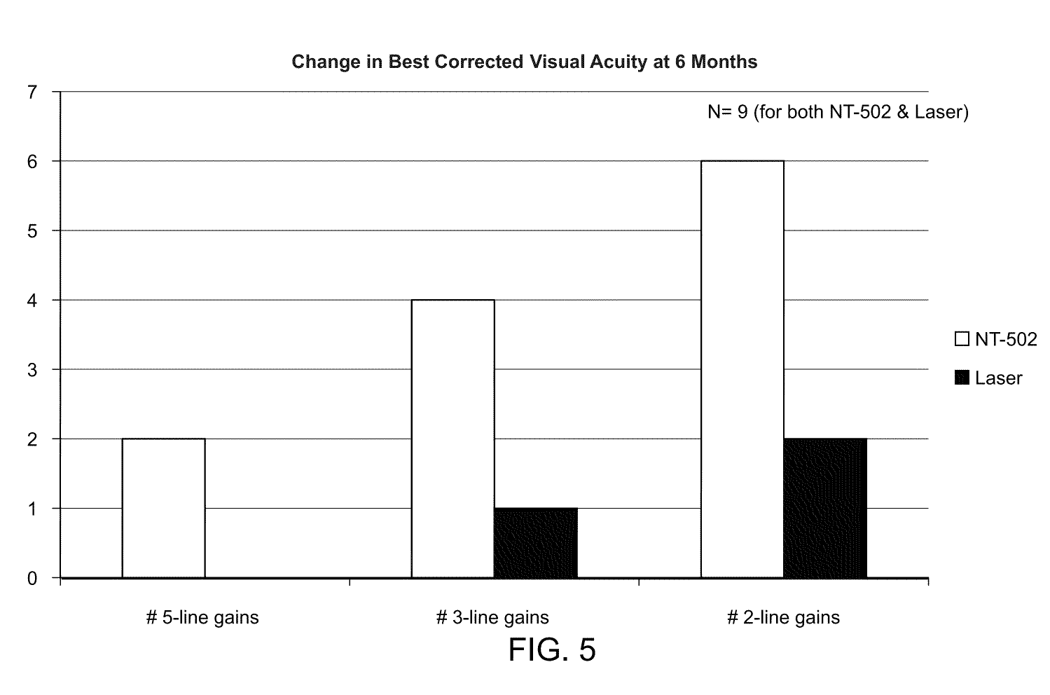

FIG. 3 shows the change in BCVA results at 1 month, 3 months, 4 months, and 6 months post-implant. The mean change in BCVA at 1, 3, 4, and 6 months is shown in FIG. 4 for NT-502 and laser treated patients. FIG. 5 also shows the change in BCVA at 6 months for both NT-502 and laser treated patients.

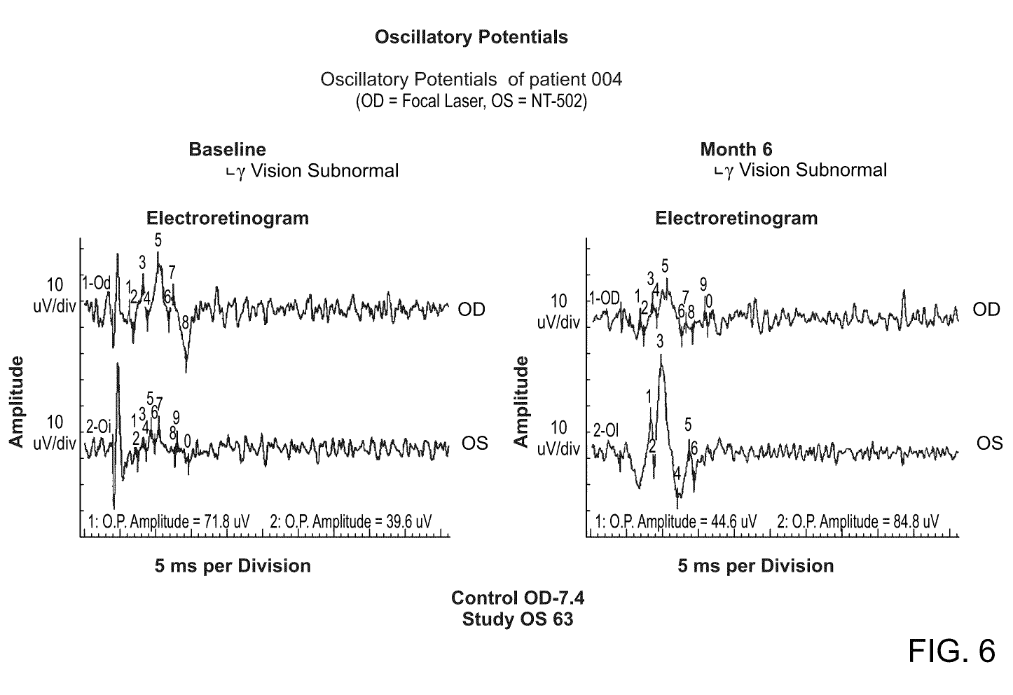

Oscillatory Potentials (OP) response reflects the function of the inner neurons and blood supply of the inner retina. In diabetic retinopathy, the OP response is reduced. An increase in OP response indicates improved microcirculation and inner neuron function. OP results at 6 months post-implant for one patient are shown in FIG. 6.

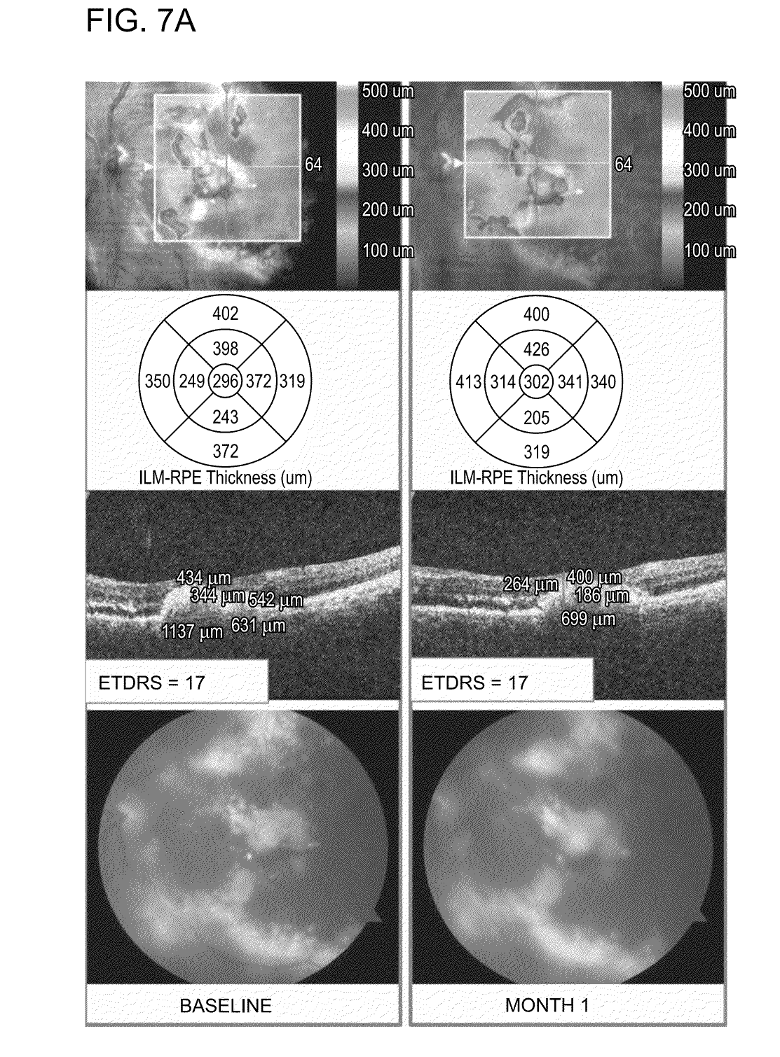

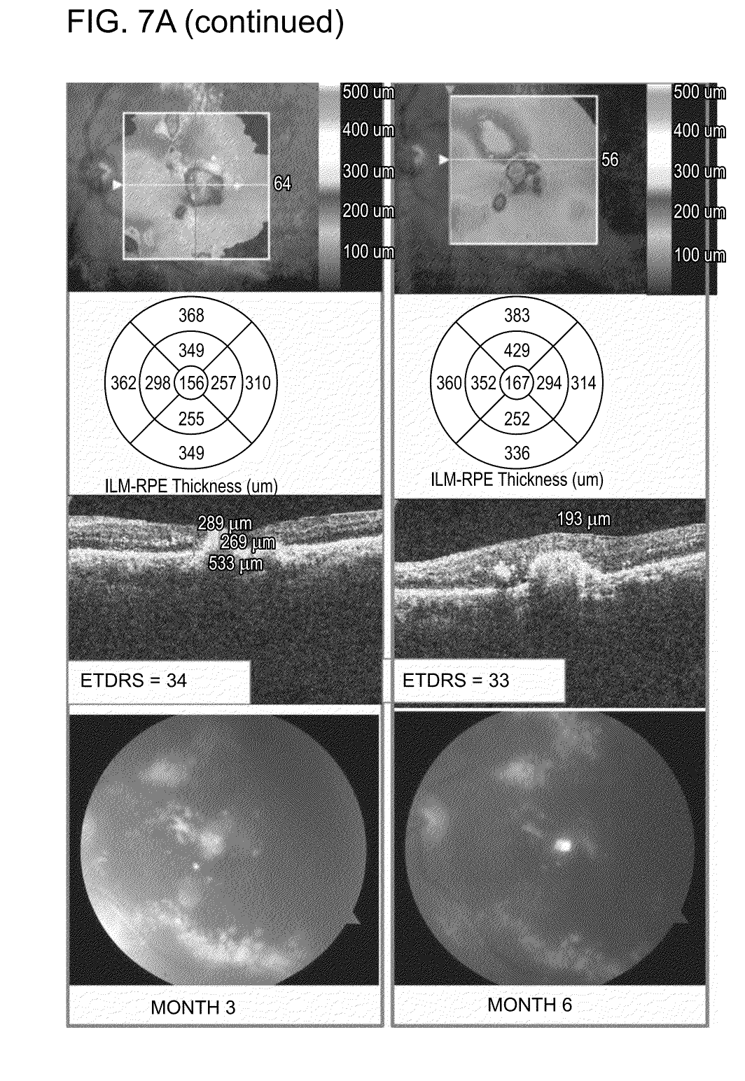

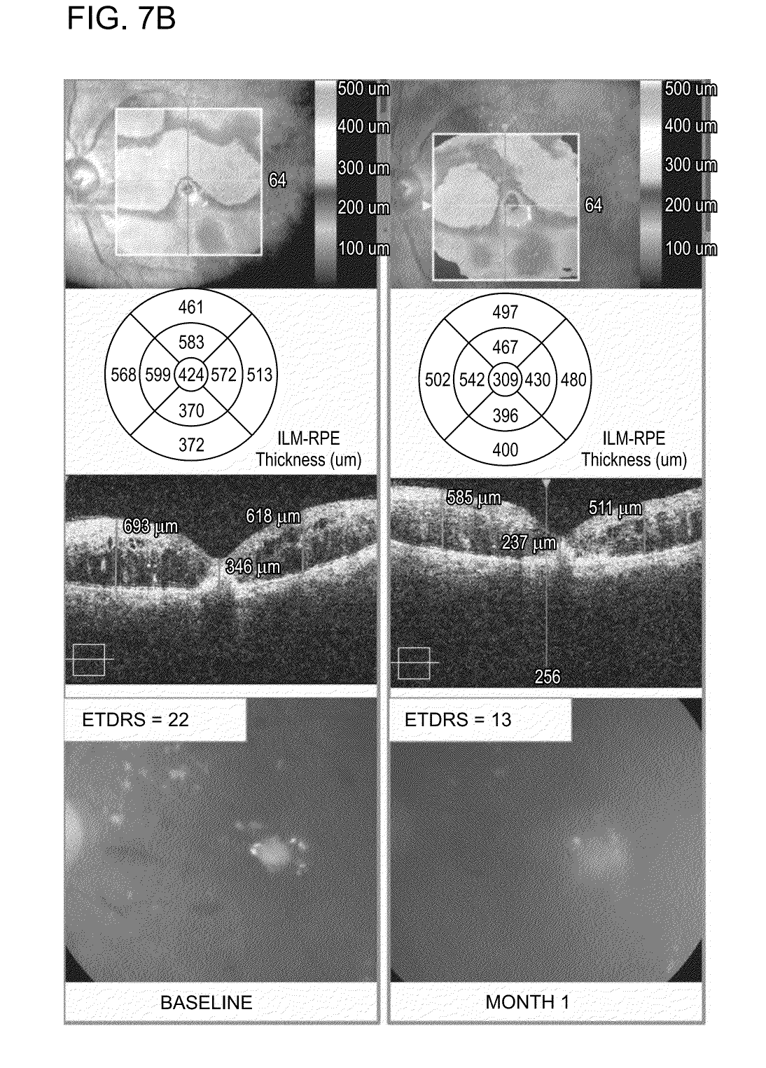

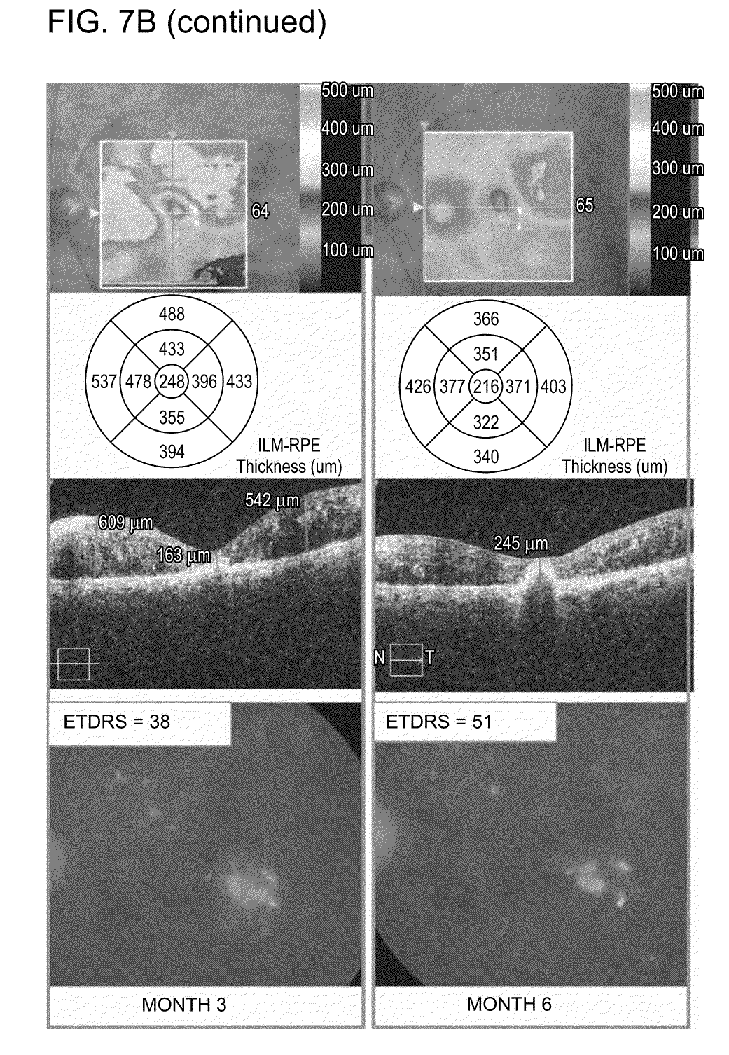

Prior to treatment, some DME patients demonstrated significant amounts of hard exudates in the eye. These hard exudates are composed of lipid and proteinaceous material that accumulates within the retina and settles in the outer retinal layer. (See, Codenotti et al., in Retina Today, Rizzo et al., eds., pages 39-40 (2010) (incorporated herein by reference)). Moreover, when deposited in the foveal region, these plaques often cause significant vision loss. (See id.) As shown in FIGS. 7A and 7B, two patients in this study (Case 002 and Case 003) showed significant amounts of hard exudates in the eye. Over time (following NT-502 treatment), the hard exudates began to breakdown and were absorbed. Such impressive clearance of hard exudates has not previously been described

The results of this study indicate that an initial trend in visual acuity improvement in eyes implanted with the NT-502 device (at least the same or better than current studies) is promising. Mild to moderate vitreous inflammation observed in some patients who responded well to transient topical steroids. Thus, as no other significant adverse events were observed, the NT-502 device had an excellent safety profile.

### EQUIVALENTS

The details of one or more embodiments of the invention are set forth in the accompanying description above. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise.

Unless defined otherwise, all technical and scientific terms used herein have the same meaning as commonly understood by one of ordinary skill in the art to which this invention belongs. All patents and publications cited in this specification are incorporated by reference. The foregoing description has been presented only for the purposes of illustration and is not intended to limit the invention to the precise form disclosed, but by the claims appended hereto.

What is claimed is:

1. An implantable cell culture device, the device comprising:
a) a core comprising one or more ARPE-19 cells that are genetically engineered to secrete PEDF; and
b) a semipermeable membrane surrounding the core, wherein the membrane permits the diffusion of PEDF therethrough.

2. The device of claim 1, wherein the cells secrete a PEDF variant.

3. The device of claim 2, wherein the PEDF variant comprises the amino acid sequence of SEQ ID NO:1.

4. The device of claim 2, wherein the PEDF variant is a biologically active fragment of PEDF.

5. The device of claim 1, wherein the core further comprises a matrix disposed within the semipermeable membrane.

6. The device of claim 5, wherein the matrix comprises a hydrogel or extracellular matrix components.

7. The device of claim 6, wherein the hydrogel comprises alginate cross-linked with a multivalent ion.

8. The device of claim 5, wherein the matrix comprises a plurality of monofilaments, wherein said monofilaments are
a) twisted into a yarn or woven into a mesh or
b) twisted into a yarn that is in non-woven strands,
and wherein the cells or tissue are distributed thereon.

9. The device of claim 8, wherein the filamentous cell-supporting matrix comprises a biocompatible material selected from the group consisting of acrylic, polyester, polyethylene, polypropylene polyacetonitrile, polyethylene terephthalate, nylon, polyamides, polyurethanes, polybutester, silk, cotton, chitin, carbon, and biocompatible metals.

10. The device of claim 1, wherein the device further comprises a tether anchor.

11. The device of claim 10, wherein the tether anchor comprises an anchor loop.

12. The device of claim 11, wherein the anchor loop is adapted for anchoring the device to an ocular structure.

13. The device of claim 12, wherein the device is implanted into the eye.

14. The device of claim 13, wherein the device is implanted in the vitreous, the aqueous humor, the Subtenon's space, the periocular space, the posterior chamber, or the anterior chamber of the eye.

15. The device of claim 1, wherein the jacket comprises a permselective, immunoisolatory membrane.

16. The device of claim 1, wherein the jacket comprises an ultrafiltration membrane or a microfiltration membrane.

17. The device of claim 1, wherein the jacket comprises a non-porous membrane material.

18. The device of claim 17, wherein the non-porous membrane material is a hydrogel or a polyurethane.

19. The device of claim 1, wherein the device is configured as a hollow fiber or a flat sheet.

20. The device of claim 1, wherein at least one additional biologically active molecule is co-delivered from the device.

21. The device of claim 20, wherein the at least one additional biologically active molecule is from a non-cellular source.

22. The device of claim 20, wherein the at least one additional biologically active molecule is from a cellular source.

23. The device of claim 22, wherein the at least on additional biologically active molecule is produced by one or more genetically engineered ARPE-19 cell in the core.

24. The device of claim 1, wherein the semipermeable membrane has a molecular weight cutoff of from 1 to 1500 kilodaltons.

25. The device of claim 1, wherein the device is a hollow fiber having an outer diameter between 200 and 350 μm and a length of between 0.4 mm and 6 mm.

26. The device of claim 25, wherein the hollow fiber is a polyether sulfone hollow fiber.

27. The device of claim 1, wherein the device has a core volume of between 1 and 3 μl.

28. The device of claim 1, wherein the device has a core volume of between 0.05 and 0.1 μl.

29. The device of claim 1, wherein the capsule contains from 10⁴ to 10⁷ cells.

30. The device of claim 1, wherein the semipermeable membrane is formed from a material selected from the group consisting of polyacrylates (including acrylic copolymers), polyvinylidenes, polyvinyl chloride copolymers, polyurethanes, polystyrenes, polyamides, cellulose acetates, cellulose nitrates, polysulfones (including polyether sulfones), polyphosphazenes, polyacrylonitriles, poly(acrylonitrile/covinyl chloride), and derivatives, copolymers and mixtures thereof.

31. A method for treating an ophthalmic disease or disorder characterized by retinal degeneration, neovascularization fluid accumulation in the eye, or any combination thereof, in a subject in need of such treatment, comprising implanting the implantable cell culture device of claim 1 into the eye of the subject and allowing PEDF to diffuse from the device into the eye, thereby treating the disease or disorder.

32. The method of claim 31, wherein the subject is a human.

33. The method of claim 31, wherein the ophthalmic disease or disorder is age-related macular degeneration, retinitis pigmentosa, diabetic macular edema, or diabetic retinopathy.

34. The method of claim 31, wherein the device is implanted intraocularly or periocularly.

35. The method of claim 31, wherein between 0.1 pg and 1000 μg per eye per patient per day of PEDF diffuse into the eye.

36. A method for inhibiting neural or retinal degradation or degeneration in a host comprising implanting the cell culture device of claim 1 into the eye of a host, wherein the device secretes a therapeutically effective amount of PEDF into the eye, thereby allowing PEDF to function as a neurotrophic or neuroprotective agent.

37. The method of claim 36, wherein the device is implanted intraocularly or periocularly.

38. A method of delivering PEDF to a recipient host, comprising implanting the implantable cell culture device of claim 1 into a target region of the recipient host, wherein the encapsulated one or more ARPE-19 cells secrete PEDF at the target region.

39. The method of claim 38, wherein the target region is selected from the group consisting of the central nervous system, including the brain, ventricle, spinal cord, and the aqueous and vitreous humors of the eye.

40. The method of claim 38, wherein between 0.1 pg and 1000 μg per patient per day of PEDF diffuses into the target region.

41. A method for inhibiting vasopermeability associated with angiogenesis, retinal disease, or a combination thereof in a host comprising implanting the cell culture device of claim 1 into the eye of a host, wherein the device secretes a therapeutically effective amount of PEDF into the eye, thereby allowing PEDF to inhibit vasopermeability.

42. A method for making the implantable cell culture device of claim 1, comprising
a) genetically engineering at least one ARPE-19 cell to secrete a PEDF polypeptide encoded by the nucleic acid sequence of SEQ ID NO:2 or SEQ ID NO:4, and
b) encapsulating said genetically modified ARPE-19 cells within a semipermeable membrane, wherein said membrane allows the diffusion of PEDF therethrough.

43. A method for making the implantable cell culture device of claim 1 comprising
a) genetically engineering at least one ARPE-19 cell to secrete a PEDF polypeptide comprising the amino acid sequence of SEQ ID NO:1, and
b) encapsulating said genetically modified ARPE-19 cells within a semipermeable membrane, wherein said membrane allows the diffusion of PEDF therethrough.


SEQUENCE LISTING (4 sequences)

SEQ ID NO: 1 - protein, 418 aa, Artificial Sequence
  M72T PEDF Variant
MQALVLLLCIGALLGHSSCQNPASPPEEGSPDPDSTGALVEEEDPFFKVPVNKLAAAVSNFGYDLYRVRSSTSPTTNVLLSPLSVATALSALSLGAEQRTESIIHRALYYDLISSPDIHGTYKELLDTVTAPQKNLKSASRIVFEKKLRIKSSFVAPLEKSYGTRPRVLTGNPRLDLQEINNWVQAQMKGKLARSTKEIPDEISILLLGVAHFKGQWVTKFDSRKTSLEDFYLDEERTVRVPMMSDPKAVLRYGLDSDLSCKIAQLPLTGSMSIIFFLPLKVTQNLTLIEESLTSEFIHDIDRELKTVQAVLTVPKLKLSYEGEVTKSLQEMKLQSLFDSPDFSKITGKPIKLTQVEHRAGFEWNEDGAGTTPSPGLQPAHLTFPLDYHLNQPFIFVLRDTDTGALLFIGKILDPRGP

SEQ ID NO: 2 - DNA, 1257 bp, Artificial Sequence
  M72T PEDF Variant cDNA
atgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcaccgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaa

SEQ ID NO: 3 - DNA, 4246 bp, Artificial Sequence
  nucleotide sequence of pKAN2
cttggtttttaaaaccagcctggagtagagcagatgggttaaggtgagtgacccctcagccctggacattcttagatgagccccctcaggagtagagaataatgttgagatgagttctgttggctaaaataatcaaggctagtctttataaaactgtctcctcttctcctagcttcgatccagagagagacctgggcggagctggtcgctgctcaggaactccaggaaaggagaagctgaggttaccacgctgcgaatgggtttacggagatagctggctttccggggtgagttctcgtaaactccagagcagcgataggccgtaatatcggggaaagcactatagggacatgatgttccacacgtcacatgggtcgtcctatccgagccagtcgtgccaaaggggcggtcccgctgtgcacactggcgctccagggagctctgcactccgcccgaaaagtgcgctcggctctgccaaggacgcggggcgcgtgactatgcgtgggctggagcaaccgcctgctgggtgcaaaccctttgcgcccggactcgtccaacgactataaagagggcaggctgtcctctaagcgtcacccctagagtcgagctgtgacggtccttacactcgagaccggtgcggccgcatttaaatactagtccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgacggccgtaattcgtaatcatgtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagccagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctaaatccttttaaattaaaaatgaagttttaaatcaatctaaagtatatatgagtaaacttggtctgacagtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccgatccccccggtacccgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcatgaacagactgtgaggactgaggggcctgaaatgagccttgggactgtgaatctaaaatacacaaacaattagaatcagtagtttaacacattatacacttaaaaattttatatttaccttagagctttaaatctctgtaggtagtttgtccaattatgtcacaccacagaagtaaggttccttcacaaagatcccaagtcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcccaagctggggatctgcaggaatcgatatcaagcttatcgataagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcagaaggtacactcttcctttttcaatattattgaagcatttatcagggttattgtctcatgagcggatacatatttgaatgtatttagaaaaataaacaaataggggttccgcgcacatttccccgaaaagtgccacctgacggcggcc

SEQ ID NO: 4 - DNA, 1257 bp, Artificial Sequence
  PEDF polynucleotide
atgcaggccctggtgctactcctctgcattggagccctcctcgggcacagcagctgccagaaccctgccagccccccggaggagggctccccagaccccgacagcacaggggcgctggtggaggaggaggatcctttcttcaaagtccccgtgaacaagctggcagcggctgtctccaacttcggctatgacctgtaccgggtgcgatccagcacgagccccacgaccaacgtgctcctgtctcctctcagtgtggccacggccctctcggccctctcgctgggagcggagcagcgaacagaatccatcattcaccgggctctctactatgacttgatcagcagcccagacatccatggtacctataaggagctccttgacacggtcactgccccccagaagaacctcaagagtgcctcccggatcgtctttgagaagaagctgcgcataaaatccagctttgtggcacctctggaaaagtcatatgggaccaggcccagagtcctgacgggcaaccctcgcttggacctgcaagagatcaacaactgggtgcaggcgcagatgaaagggaagctcgccaggtccacaaaggaaattcccgatgagatcagcattctccttctcggtgtggcgcacttcaaggggcagtgggtaacaaagtttgactccagaaagacttccctcgaggatttctacttggatgaagagaggaccgtgagggtccccatgatgtcggaccctaaggctgttttacgctatggcttggattcagatctcagctgcaagattgcccagctgcccttgaccggaagcatgagtatcatcttcttcctgcccctgaaagtgacccagaatttgaccttgatagaggagagcctcacctccgagttcattcatgacatagaccgagaactgaagaccgtgcaggcggtcctcactgtccccaagctgaagctgagttatgaaggcgaagtcaccaagtccctgcaggagatgaagctgcaatccttgtttgattcaccagactttagcaagatcacaggcaaacccatcaagctgactcaggtggaacaccgggctggctttgagtggaacgaggatggggcgggaaccacccccagcccagggctgcagcctgcccacctcaccttcccgctggactatcaccttaaccagcctttcatcttcgtactgagggacacagacacaggggcccttctcttcattggcaagattctggaccccaggggcccctaa